(19) United States
(12) Patent Application Publication
(10) Pub. No.: US 2021/0052563 A1
(43) Pub. Date: Feb. 25, 2021

(54) PROTON PUMP INHIBITORS AND METHODS OF USE IN CHEMORADIATION-INDUCED TISSUE INFLAMMATION AND SCARRING

(71) Applicants: Baylor College of Medicine, Houston, TX (US); The Methodist Hospital d/b/a Houston Methodist, Houston, TX (US)

(72) Inventors: Yohannes T. Ghebre, Houston, TX (US); Michelle S. Ludwig, Houston, TX (US); Mark David Bonnen, Houston, TX (US); Pavan Mukesh Jhaveri, Houston, TX (US)

(21) Appl. No.: 16/975,257

(22) PCT Filed: Feb. 28, 2019
(86) PCT No.: PCT/US2019/020117

§ 371 Date: Aug. 24, 2020

Related U.S. Application Data
(60) Provisional application No. 62/636,284, filed on Feb. 28, 2018.

(51) Int. Cl. A61K 31/4439; A61K 33/30; A61K 33/38; A61K 31/661; A61K 45/06; A61K 9/00; A61P 17/00

(52) U.S. Cl. CPC A61K 31/4439; A61K 33/30; A61K 33/38; A61P 17/00; A61K 45/06; A61K 9/0014; A61K 31/661

(57) ABSTRACT

Embodiments of the disclosure include methods and compositions related to treatment or prevention of cancer therapy-induced tissue inflammation, dermatitis, and/or scarring. In particular embodiments one or more proton pump inhibitors are provided to an individual before, during, and/or after receiving anticancer therapy. In some embodiments one or more proton pump inhibitors are provided to an individual before, during, and/or after having a health conditions, allergies, genetic factors and/or exposure to one or more irritants.

This application claims priority to U.S. Provisional Application No. 62/636,284, filed Feb. 28, 2018, which is incorporated by reference herein in its entirety.

### STATEMENT REGARDING FEDERALLY SPONSORED RESEARCH OR DEVELOPMENT

This invention was made with government support under K01HL118683 awarded by National Institutes of Health. The government has certain rights in the invention.

### TECHNICAL FIELD

Embodiments of the disclosure include at least the fields of cell biology, molecular biology, biochemistry, pharmacology, and medicine.

### BACKGROUND

Chemotherapy, with or without radiation, is a standard of care approach for the treatment of various cancers. Unfortunately, many of the commonly-used chemotherapeutic agents including cisplatin, doxorubicin, 5-fluorouracil and paclitaxel as well as epidermal growth factor receptor (EGFR) inhibitors, tyrosine kinase inhibitors (TKIs), and others are associated with tissue inflammation and fibrosis that on one hand promotes tumor cell proliferation, angiogenesis and cancer metastasis, and on the other hand interrupts the treatment regimen due to excessive inflammatory response.

Such chemoradiation-induced fibro-inflammatory conditions affect several tissues and organs, including the lungs (pneumonitis), mucous membrane (mucositis), skin (dermatitis), rectum (proctitis), intestine (enteritis), esophagus (esophagitis) and blood vessels (vasculitis). As a result, cancer survivors often suffer from these often painful and sometimes disfiguring complications of chemoradiation.

Dermatitis, for example, is inflammation of the skin that is characterized by subcutaneous and vascular damage involving endothelial and epidermal basal cells. The disease has a very high incidence rate (up to 95%) in patients who receive chemoradiation for breast cancer, sarcoma, and head-and-neck cancer. In most of these cases, the inflammation subsides with mild erythema. However, about 20 to 25% of the patients develop severe skin reactions including moist desquamation and ulceration which can lead to necrosis and scarring. Unfortunately, these complications often interrupt the treatment plan and threaten the relapse of underlying cancer. Topical corticosteroids have been developed to treat dermatitis. However, their use is limited due to the risk of cutaneous atrophy and secondary skin infection with long term use. Accordingly, there is an unmet clinical need for this indication.

Radiation therapy (RT) is part of the treatment regimen for many patients with cancers of the breast, lung, head and neck, and other solid tumors (Mendelsohn et al., 2002). As a result of this medical intervention, the survival and quality of life of cancer patients has been steadily improving. Unfortunately, many cancer survivors treated with large doses of RT often suffer from off-target effects including the development of widespread skin inflammation (radiation dermatitis) and progressive fibrosis that could lead to painful and disfiguring scar in the face, chest, head and neck area. Acute radiation dermatitis is characterized by excessive skin inflammation that involves epidermal, dermal and vascular tissues and occurs in up to 95% of patients who receive RT (Chan et al., 2014). Up to 25% of the patients develop severe skin reactions including moist desquamation and ulceration which can lead to necrosis and scarring. Unfortunately, these complications often interrupt the treatment plan and threaten the relapse of underlying cancer including tumor re-growth, metastasis and cancer-related death (Chen et al., 2000; McCloskey et al., 2009; Putora et al., 2012).

Symptoms of radiation dermatitis vary in onset and duration depending on the total radiation dose delivered. According to the National Cancer Institute Common Toxicity Criteria-Adverse Events (NCI-CTCAE) (US Department of Health and Human Services. Common Terminology Criteria for Adverse Events Version 4.0, 2012) and the Radiation Therapy Oncology Group (RTOG) (Cox et al., 1995; Trotti et al., 2000) toxicity scoring system, mild dermatitis (Grade 1), characterized by mild redness (erythema), hyperpigmentation, itching, epidermal thickening (hyperkeratosis) or dry desquamation, appears shortly after initiation of RT. Moderate dermatitis (Grade 2) occurs within two weeks of the completion of therapy and manifests painful and intense erythema, loss of hair from the root (epilation), epidermal necrosis, blisters and edema. In severe dermatitis (Grade 3 and 4), moist desquamation occurs prominently and may lead to persistent inflammation, full-thickness skin necrosis and severely painful ulceration that is prone to infection. The acute and milder skin effects occur almost immediately at radiation doses between 2 and 40 Gray (2-40 Gy) whereas chronic effects occur several months-to-years after exposure to high radiation dose (>45 Gy) and typical skin changes include necrosis, atrophy, scarring and spider veins (telangiectasia) (Brown et al., 2011). These structural and functional impairments to the skin are driven in part by the exquisite sensitivity of hair follicle stem cells, basal keratinocytes and melanocytes to radiation. Fractionated doses of radiation repeatedly injure these resident skin cells resulting in impairment to self-renew and repair tissue damage (Mendelsohn et al., 2002). In addition, radiation ionizes cellular and tissue water to promote the production of reactive oxygen species (ROS) and adducts that are involved in DNA damage (Lopez et al., 2005; Gamullin et al., 2007; Lomax et al., 2013). Furthermore, recruitment of circulating inflammatory cells to the local vasculature and increased levels of inflammatory cytokines and chemokines (Brach et al., 1993; Muller and Meineke, 2007; Mukherjee et al., 2014; Okunieff et al., 2006) (e.g. TNFα, IL1β, VCAM1 and ICAM1) exacerbates the injury and compromises the integrity of the epidermal and dermal layers of the skin leading to increased susceptibility to infection, delayed wound healing, fibrous thickening and irreversible scarring.

Several approaches have been evaluated for the prevention and treatment of severe radiation dermatitis. Among the general preventative strategies, hyaluronic acid-based formulations, petroleum-based ointments, hydrogel-based dressings, aloe vera gel, honey, curcumin, sorbolene cream, wheatgrass extract cream, almond oil, trolamine, calendula and sucralfate have been evaluated in clinical studies (Chan et al., 2014; Sitton, 1992; Campbell and Illingworth, 1992; Roy et al., 2001; Wells et al., 2004; Elliott et al., 2006; Richardson et al., 2005; Macmillan et al., 2007). However, the use of almost all of these agents is not recommended either due to lack of efficacy or insufficient clinical data (Chan et al., 2014). Among steroid-based products, mometasone furoate (0.1%), betamethasone (0.1%) and hydrocortisone (1%) have been extensively studied in clinical trials (Hainan, 1962; Glees et al., 1979; Rostrom et al., 2001; Schmuth et al., 2002; Omidvari et al., 2007; Miller et al., 2011; Ulff et al., 2013; Ho et al., 2018). However, the actual use of topical corticosteroid for radiation dermatitis is limited due to the risk of epidermal thinning, cutaneous atrophy, stretch marks (striae), allergy and secondary skin infection (cellulitis) (Coondoo et al., 2014). Accordingly, there is an unmet clinical need to develop safe and effective topical formulations.

The present disclosure satisfies a long-felt need in the art of providing therapies for chemotherapy- and/or radiation therapy-induced tissue inflammation and scarring, among other medical conditions, for example.

### BRIEF SUMMARY

Embodiments of the present disclosure are directed to methods and compositions for the treatment or prevention of one or more dermatological (relating to the skin including the epidermis, dermis, or both) and other conditions, including at least chemoradiation-induced inflammation and scarring (fibrosis); chemoradiation-induced diarrhea or colitis, radiation-induced dermal inflammation and fibrosis; scleroderma; Mixed Connective Tissue Disease (MCTD); rheumatic diseases including Rheumatoid Arthritis (RA); lupus; polymyositis; dermatomyositis; atopic dermatitis; seborrheic dermatitis; Raynaud Disease; oral mucositis; scarring of any kind or cause; keloids; chloracne; acne; wrinkled skin; aging skin; oxidative stress of skin; sunburn; photodamage; skin barrier protection; skin barrier photoprotection; skin cancer; psoriasis; vitiligo; allergic dermatitis; atopic dermatitis; inflammatory skin conditions of any kind or cause; wound healing; burns of any kind (thermal, chemical, freeze), prostatitis, proctitis, bladder and urinary tract inflammation, plastic or cosmetic surgery with skin grafting or transplant, graft vs host disease of the skin, for example after stem cell transplant and/or aphthous ulcers (canker sores) as examples.

Embodiments of the disclosure include methods and compositions for treatment or prevention of one or more indications where one or more inflammatory markers (as examples only, C-reactive protein (CRP) and tumor necrosis factor-α) are involved, such as markers that are elevated with respect to that of the general population. Methods and compositions of the disclosure concern treatment and prevention of inflammation and/or fibrosis related to any medical condition.

In particular embodiments, one or more proton pump inhibitors (PPIs) are utilized for chemoradiation-induced inflammation and scarring and/or radiation-induced dermal inflammation and fibrosis. In this regard, the PPIs may suppress chemoradiation-induced inflammation and scarring and/or radiation-induced dermal inflammation and fibrosis, as well as enhance the efficacy of the respective therapies by increasing the sensitivity of the underlying tumor cells.

The PPIs may be formulated for administration by particular routes. In particular embodiments, the PPIs are not utilized for gastritis or gastritis-related purposes. In specific embodiments, the methods and compositions of the disclosure encompass reformulations of proton pump inhibitors (PPI) that had been used to treat gastritis for new anti-inflammatory/antifibrotic indications and in new delivery modalities. Examples of PPIs include Omeprazole, Lansoprazole, Dexlansoprazole, Esomeprazole, Pantoprazole, Rabeprazole, Ilaprazole, and a combination thereof.

The methods and compositions may be utilized for any kind of individual, including all mammals, such as human, dog, cat, horse, and so forth. In specific embodiments, the PPIs are present in liquid, troche, and suppository formulations, as examples.

In one embodiment, there is a method of treating or preventing chemotherapy- and/or radiation therapy-induced tissue inflammation, dermatitis, fibrosis, and/or scarring in an individual, comprising the step of administering to the individual an effective amount of one or more PPIs. The administering may occur systemically or locally. Local administering may be to the lungs, mucous membrane, skin, rectum, intestine, esophagus and/or blood vessels. A proton pump inhibitor may be formulated as a liquid, troche, suppository, cream, solid, tablet, pill, aerosol, gel, film, or foam. The one or more PPIs may be administered prior to, during, and/or subsequent to administration of chemotherapy, radiation therapy, or both. Specific PPIs include but are not limited to Omeprazole, Lansoprazole, Dexlansoprazole, Esomeprazole, Pantoprazole, Rabeprazole, Ilaprazole, or a combination thereof. The chemotherapy may be of any kind including but not limited to bleomycin, carboplatin, cisplatin, doxorubicin, etoposide, mitomycin, cetuximab, gemcitabine, capecitabine, 5-fluorouracil, paclitaxel, or a combination thereof. Methods of the disclosure include those that further comprise the step of administering any cancer therapy including chemotherapy or radiation; one or more receptor tyrosine kinase inhibitors; one or more monoclonal antibodies; one or more immune checkpoint inhibitors; or a combination thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

FIG. 1 provides an example of an illustration wherein PPIs activate HO1 by inducing nuclear translocation of Nrf2 either through phosphorylation of ERK1, Nrf2 itself and/or attacking the sulfhydryl group in Keap1 and disassociating Keap1-Nrf2 complex. P=phosphorylation; PPIs=proton pump inhibitors; HO1=heme oxygenase; Nrf2=nuclear factor-like 2; ARE=antioxidant response elements.

FIG. 2 provides a representative chromatography (LC-MS) data showing stability of esomeprazole cream (coined dermaprazole”) after 30 days of formulation and storage. The single peak shows intact (i.e. degradation-free) demaprazole.

FIG. 3 demonstrates protein expression data showing induction of Nrf2 by dermaprazole (1-2%) in nuclear fraction of cell extracts from human 3D skin model at baseline (“No RT”) and after 14 gray irradiation. The protein levels of histone H3 are shown as loading control. Anti-Nrf2 Rabbit monoclonal Antibody (Abcam; ab62352,1:250) and Rabbit polyclonal to Histone H3 (Abcam; ab1791; 1:3000) are used. 1% hydrocortisone (“Steroid”) are used as controls. RT=radiation treatment.

FIG. 4 demonstrates protein expression data showing induction of HO1 by dermaprazole (1-5%) in total cell extracts from human 3D skin model at baseline (“No RT”) and after 14 gray irradiation. The protein levels of GAPDH are shown as loading control. Anti-HO1 Rabbit monoclonal Antibody (Enzo; BML-HC3001,1:250) and GAPDH monoclonal antibody (ThermoFisher; MA5-15738,1:3000) are used. 1% hydrocortisone (“Steroid”) are used as controls. RT=radiation treatment; GAPDH=glyceraldehyde 3-phosphate dehydrogenase.

FIG. 5 provides protein expression data showing activation/phosphorylation of ERK1/2 (pERK1/2) by dermaprazole in irradiated (14 gray) human 3D skin tissue. The protein levels of GAPDH is shown as loading control. Anti-pERK1/2 Rabbit monoclonal Antibody (Cell Signaling Technology; 4370, 1:2000); and GAPDH monoclonal Antibody (ThermoFisher; MA5-15738, 1:3000) are used as primary Antibodies. HRP-conjugated donkey Anti-Rabbit (GE Healthcare; NA934V, 1:5000) is used as secondary Antibody. ERK=extracellular signal-regulated kinase.

FIG. 6 demonstrates gene expression data showing induction of Nrf2 and HO1 by dermaprazole in human 3D skin model (EpiDermFT; MatTekCorporation) following 14 gray irradiation. VEH is placebo cream lacking esomeprazole. 1% hydrocortisone (“Steroid”) which is currently used to treat dermatitis is used as a control. *p<0.05 compared to VEH control. HO1=heme oxygenase; Nrf2=nuclear factor-like 2; VEH=vehicle.

FIG. 7 shows regulation of the NOS/DDAH pathway by PPIs. PPIs directly inhibit DDAH enzymatic activity resulting in accumulation of the endogenous substrate ADMA. ADMA is a competitive NOS inhibitor and limits the production of reactive oxygen and nitrogenous species resulting in reduced tissue inflammation and fibrosis. Physiologically, oxidation of L-Arginine in the presence of NOS generates nitric oxide. iNOS=inducible nitric oxide synthase; DDAH=dimethylarginine dimethylaminohydrolase; ADMA=asymmetric dimethylarginine; PPIs=proton pump inhibitors.

FIG. 8 shows gene expression profiling of the transcription factor erythroid 2-related factor 2 (Nrf2) and the antioxidant enzyme heme oxygenase 1 (HO1) in irradiated EpidermFT tissue homogenates from a 3D human skin model. The EpidermFT was exposed to various strengths of dermaprazole, vehicle cream or the steroid hydrocortisone (1%) for 24 hours. Fold change normalized to the vehicle control is shown. Data is from duplicate experiments. *p<0.05 compared to the expression of Nrf2 in the vehicle and +p<0.05 compared to the expression of HO1 in the vehicle group.

FIG. 9 provides western blot analysis of erythroid 2-related factor 2 (Nrf2) and heme oxygenase 1 (HO1) proteins in homogenates derived from irradiated EpidermFT tissues. Nuclear and cytoplasmic proteins were fractionated and Nrf2 was probed in the nuclear fraction using rabbit anti-Nrf2 Antibody (Abcam; ab62352, 1:250) and the house keeping gene histone H3 was probed using rabbit anti-histone H3 Antibody (Abcam; ab1791, 1:3000). HO1 was probed in the cytoplasmic fraction using rabbit anti-HO1 (Enzo; BML-HC3001, 1:500) and GAPDH was detected using mouse anti-GAPDH Antibody (ThermoFisher; MA5-15738, 1:5000). The secondary Antibody was anti-rabbit monoclonal (GE Healthcare; NA934V, 1:5000) or anti-mouse monoclonal (1:5000). The data shows that dermaprazole upregulates the protein expression of Nrf2 and HO1. VEH=vehicle.

FIG. 10 shows western blot analysis of heme oxygenase 1 (HO1) protein in homogenates derived from unirradiated EpidermFT tissue. Dermaprazole (1-2%) was applied on the tissue topically and the viable EpidermFT tissue was incubated at 370 C/5% CO2 for 24 hours. HO1 was probed using rabbit anti-HO1 (Enzo; BML-HC3001, 1:500) and GAPDH was detected using mouse anti-GAPDH Antibody (ThermoFisher; MA5-15738, 1:5000). The secondary Antibody was anti-rabbit monoclonal (GE Healthcare; NA934V, 1:5000). The data shows that dermaprazole upregulates HO1 expression in the absence of ionizing radiation. VEH=vehicle.

FIG. 11 demonstrates topical application of the PPI esomeprazole improves skin appearance in a fractionated radiation-induced model of dermatitis. Mice were irradiated (2×15 Gy) on Days 0 & 7. Topical esomeprazole (i.e. dermaprazole), vehicle (base) cream, or the corticosteroid hydrocortisone were applied once a day on the indicated days (D1-D30 for prophylactic group & D10-D30 for therapeutic group). Representative images from the same animals are shown.

FIG. 12 demonstrates H&E stain showing that topical application of the PPI esomeprazole improves skin histology in a fractionated radiation-induced model of dermatitis. Mice were irradiated (2×15 Gy) on Days 0 & 7. Topical esomeprazole (i.e. dermaprazole), vehicle (base) cream, or the steroid hydrocortisone (1.0%) were applied once a day on the indicated days (D1-D30 for prophylactic group & D10-D30 for therapeutic group). Dermal fibrosis is observed in the Vehicle group and ulceration is seen by day 30 in the steroid-treated group. Representative images are shown at 20× magnification. The scale bar shown in red line in the vehicle group at day 16 is 50 μm and applies to all the images.

FIG. 13 demonstrates Masson's trichrome stain showing that topical application of the PPI esomeprazole inhibits skin fibrosis in a fractionated radiation-induced model of dermatitis. Mice were irradiated (2×15 Gy) on Days 0 & 7. Topical esomeprazole (i.e. dermaprazole), vehicle (base) cream, or the steroid hydrocortisone (1.0%) were applied once a day on the indicated days (D1-D30 for prophylactic group & D10-D30 for therapeutic group). Increased collagen deposition (blue stain) is observed in the vehicle and steroid groups. Representative images are shown at 20× magnification. The scale bar shown in black line in the vehicle group at day 16 is 50 μm and applies to all the images.

FIGS. 14A and 14B. FIG. 14A provides H&E stain showing that topical application of the PPI esomeprazole improves skin histology in an animal model of bleomycin-induced skin inflammation and fibrosis (scleroderma model). The vehicle (aquaphor) group shows epidermal thickening (red line) & the steroid group shows loss of epidermal layer (arrow). FIG. 14B provides Masson's Trichrome stain (blue) showing that topical application of esomeprazole reduces dermal fibrosis in the same animal model.

FIG. 15 shows skin permeation/retention of dermaprazole ex vivo using Franz Diffusion Cell technique. Mouse abdominal skin was exposed to various strengths of dermaprazole and the release of esomeprazole from the dermaprazole cream (Y-axis) was measured over time (X-axis). Data shown is Mean value from duplicate experiments. *p<0.05 compared to 1% dermaprazole at the corresponding time point.

FIGS. 16A and 16B demonstrate that Dermaprazole reduces histopathological changes in the dermal tissue of radiation-induced dermatitis model. H&E stained skin tissues were evaluated by board-certified dermatopathologist who was unaware of the treatment groups. Topical dermaprazole significantly reduced inflammation, epidermal thickening and parakeratosis at day 16 (FIG. 16A), as well as inflammation, epidermal thickening, ulcer, necrosis and parakeratosis by day 30 (FIG. 16B) of the study. Scoring is based on the National Cancer Institute's Common Toxicity Criteria-Adverse Events (NCI-CTCAE) and the Radiation Therapy Oncology Group (RTOG). *p<0.05 compared to steroid (1% hydrocortisone) treated controls.

FIGS. 17A-17H demonstrates that Dermaprazole regulates radiation-induced changes in the expression of pro-inflammatory molecules in the dermal tissue of radiation dermatitis model. Quantitative RT-PCR data showing the gene expression profile of TNFα (FIG. 17A), IL1β (FIG. 17B), IL6 (FIG. 17D), iNOS (FIG. 17E), NFκB (FIG. 17C), TLR4 (FIG. 17G), VCAM1 (FIG. 17F) and ICAM1 (FIG. 17H) is shown. Data is Mean±SEM from duplicate experiments. *p<0.05 compared to steroid treated control. Pro=Prophylactic; Ther=Therapeutic

FIG. 18 shows that Dermaprazole reduces collagen thickening/fibrosis in a mouse model of radiation-induced dermatitis. Masson's trichrome stained skin tissues were evaluated by board-certified dermatopathologist who was unaware of the treatment groups. Topical dermaprazole significantly reduced dermal fibrosis compared to the vehicle or steroid (1% hydrocortisone) group at day 30 (*p<0.05). +p<0.05 compared to the steroid group. Scoring is based on the National Cancer Institute's Common Toxicity Criteria-Adverse Events (NCI-CTCAE) and the Radiation Therapy Oncology Group (RTOG).

FIGS. 19A-19F shows that Dermaprazole regulates radiation-induced changes in the expression of profibrotic molecules in the dermal tissue of radiation dermatitis model. Quantitative RT-PCR data showing the gene expression profile of TGFβ (FIG. 19A), collagen 1 (Col 1) (FIG. 19B), collagen 3 (Col 3) (FIG. 19D), Collagen 5 (Col 5) (FIG. 19E), fibronectin (FN1) (FIG. 19C) and DDAH1 (FIG. 19F) is shown. Data is Mean±SEM from duplicate experiments. *p<0.05 compared to steroid treated control. Pro=Prophylactic; Ther=Therapeutic

FIGS. 20A-20F demonstrates that Dermaprazole temporally regulates radiation-induced changes in the expression of oxidative stress-related genes in the dermal tissue of radiation dermatitis model. Quantitative RT-PCR data showing the gene expression profile of HO1 (FIG. 20A), NADPH oxidase 2 (NOX2) (FIG. 20B) and NADPH oxidase 4 (NOX4) (FIG. 20C) at the anticipated disease peak time (Day16) and at the study completion (Day30) (FIG. 20D for HO1; FIG. 20E for NOX2; FIG. 20F for NOX4) is shown. Data is Mean±SEM from duplicate experiments. *p<0.05 compared to steroid treated control. Pro=Prophylactic; Ther=Therapeutic

FIG. 21 provides a representative LC-MS chromatogram of dermaprazole: The left panel shows that dermaprazole (1%) prepared fresh (Day 0) or stored at room temperature for 18 days (Day 18) or 32 days (Day 32) post-formulation. The data shows that esomeprazole retains its integrity after formulation into a cream & storage at ambient temperature for 1 month. The right panel shows typical chromatogram of unformulated esomeprazole powder as a reference. The Y-axis shows esomeprazole molecule counts per 100,000 and the X-axis shows acquisition time in minutes.

FIG. 22 provides an Atomic Force Microscopy (AFM) scan showing the topography of dermaprazole cream (left) in comparison to cream only control (right). The arrows in the left panel show putative drug particles. Dermaprazole appears to be more stiffer but less adhesive than the control cream. Scale=5 μm×5 μm.

FIG. 23 shows western blot analysis of Kelch-like ECH-associated protein 1 (Keap1) in homogenates derived from irradiated EpidermFT tissue. Keap1 was probed using mouse anti-Keap1 monoclonal Antibody (Abcam; ab119403, 1:1000) and the house keeping gene β-actin (ACTB) was detected using rabbit anti-ACTB Antibody (Sigma; A2066, 1:1500). Rabbit monoclonal Antibody (GE Healthcare; NA934V, 1:5000) was used as secondary Antibody. The data shows that the protein expression of Keap1 was not affected by dermaprazole.

FIG. 24 shows that topical application of dermaprazole improves dermatitis scoring in a mouse model of fractionated radiation-induced dermatitis. Animals were irradiated 2×15 Gy on days 0 and 7, and treated with dermaprazole in a prophylactic (P) or therapeutic (T) course. Vehicle (base) cream and steroid (1% hydrocortisone) treatment were included as controls. The degree of dermatitis was scored by a blinded dermatologist using CTCAE criteria: 0=normal skin appearance; 1=mild erythema; 2=moderate-to-severe erythema; 3=desquamation of 25-50% of irradiated area; 4=desquamation of >50% irradiated area; and 5=frank ulcer.

FIG. 25 shows that topical application of the PPI esomeprazole favorably modifies skin remodeling in a mouse model of bleomycin-induced dermal fibrosis (i.e. Scleroderma model). Base cream (Vehicle) and Mometasone Furoate (0.1%) are used as controls, and topical esomeprazole (1%) was used to treat animals for 1 week.

FIG. 26 demonstrates immunohistochemical staining of irradiated skin tissue for CD11b and F4/80 showing that topical application of the PPI esomeprazole inhibits the pro-inflammatory markers. Mice were irradiated on Days 0 & 7. Topical esomeprazole (i.e. dermaprazole), vehicle (base) cream, or the steroid hydrocortisone (1.0%) were applied once a day on the indicated days (D1-D30 for prophylactic group & D10-D30 for therapeutic group). Increased number of inflammatory cells (neutrophils for CD11b and macrophages for F4/80; arrows) are observed in the vehicle and steroid groups. Representative images are shown at 40× magnification. The scale bar shown in red line in the vehicle group is 50 μm and applies to all the images.

FIG. 27 provides an example of a mouse model of radiation-induced dermatitis: A) measurement of total body weight over time and B) organ weights for the heart, lungs, liver and kidneys normalized to the respective body weights at the time of necropsy is shown. Lungs and kidneys weight represents combined total weight for the left and right tissues. Treatment with dermaprazole in a prophylactic (“DERM-P”) or therapeutic (DERM-T″) course did not have adverse effect on the body or organ weights. Data is expressed as Mean±SEM.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. Some embodiments of the disclosure may consist of or consist essentially of one or more elements, method steps, and/or methods of the disclosure. It is contemplated that any method or composition described herein can be implemented with respect to any other method or composition described herein. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “administered” or “administering”, as used herein, refers to any method of providing a composition to an individual such that the composition has its intended effect on the individual. For example, one method of administering is by a direct mechanism such as, local tissue administration, transdermal patch, topical, etc.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term “preventing” as used herein refers to the methods that avert a medical condition from occurring in an individual, including averting the onset of at least one symptom of the medical condition.

The term “subject” or “individual”, as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. As a non-limiting example, an effective amount is an amount sufficient to promote formation of a blood supply sufficient to support the transplanted tissue. As another non-limiting example, an effective amount is an amount sufficient to promote formation of new blood vessels and associated vasculature (angiogenesis) and/or an amount sufficient to promote repair or remodeling of existing blood vessels and associated vasculature. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

I. Examples of Methods of Use

Embodiments of the disclosure concern the use of one or more proton pump inhibitors (PPIs) that are effective for a medical condition other than gastritis or gastritis-related purposes. In particular embodiments the methods are for medical conditions in which the skin, epidermis, and/or dermis is directly or indirectly affected. The methods encompass medical conditions in which the skin, epidermis, dermis and/or mucus membranes are affected because of exposure to one or more external conditions and/or is affected because of internal causes, such as a genetic or other cause. In conditions in which the cause of the medical condition is external, the methods may be utilized prior to exposure to the external condition, during exposure to the external condition, and/or after exposure to the external condition. In conditions in which the cause is internal, such as genetic or other causes, the methods may be utilized prior to onset of a symptom of the condition and/or after onset of symptom of the condition. In situations in which the individual is predisposed to have the medical condition, the individual may prophylactically be subject to methods of the disclosure. The methods include treatment or prevention for any inflammation-related and/or fibrosis-related medical condition. In specific embodiments, the methods provide therapy or prophylaxis to the skin, dermis, epidermis, and/or mucus membranes.

In specific embodiments, the external cause of the medical condition is environmental, such as exposure to chemical(s), radiation of any kind, and/or one or more pathogens. In particular embodiments, the individual is in need of treatment because of exposure to radiation or chemoradiation. The individual may be in need of prevention because they will be exposed to radiation or chemoradiation. As an example, radiation therapy (RT) is a mainstream strategy in the treatment of several cancer types that are surgically unresectable. Unfortunately, cancer survivors often suffer from the unintended consequences of RT, including the development of severe skin inflammation (dermatitis) that may progress into fibrosis. These morbid complications often enforce the interruption of RT and threaten the relapse of underlying cancer. Current treatment options for radiation dermatitis are suboptimal and compel the need to develop safe and effective therapies. Any skin condition related to cancer or cancer treatment may be treated or prevented with one or more PPIs, including at least head and neck cancer, skin cancer, thyroid cancer, lung cancer, breast cancer, colon cancer, liver cancer, brain cancer, blood cancer, kidney cancer, stomach cancer, testicular cancer, ovarian cancer, endometrial cancer, spleen cancer, and so on.

In the present disclosure, an example of a topically formulated PPI is shown to be effective in suppressing radiation-induced dermatitis in part through stimulation of antioxidant defense mechanisms such as the erythroid 2-related factor 2 (Nrf2) and heme oxygenase 1 (HO1) pathway, and effective for inhibition of classic pro-inflammatory molecules to control the oxidative stress, inflammation, and fibrosis that are perpetrated by ionizing radiation.

In particular, the biophysical property of topically-formulated esomeprazole (termed dermaprazole) was assessed and a study was performed to evaluate its efficacy in a 3D human skin model and in a mouse model of radiation-induced dermatitis. For the 3D model, activation of the Nrf2-HO1 pathway was assessed in the presence or absence of dermaprazole. For the animal study, X-rays were used to induce dermatitis in the flank of mice. The animals were treated with 1% or 2% dermaprazole in a prophylactic or therapeutic course. In a 3D human skin model, it was determined that dermaprazole induced nuclear translocation of Nrf2 and significantly upregulated HO1 gene and protein expression. The animal study demonstrated that dermaprazole improved macroscopic appearance of the irradiated skin including accelerated healing of the radiation-induced wounds. Histopathology data corroborated the photographic evidence and confirmed that both prophylactically and therapeutically administered dermaprazole conferred potent anti-inflammatory and antifibrotic effects, with significant reductions in the degree of ulceration, necrosis, inflammation and fibrosis. Gene expression data showed that dermaprazole significantly downregulated several pro-oxidant, pro-inflammatory and profibrotic genes.

Therefore, as an example of a PPI, the topical formulation of the FDA-approved generic drug esomeprazole is highly effective in attenuating radiation-induced skin inflammation and fibrosis. Subsequent mechanistic studies showed that dermaprazole activated the Nrf2-HO1 pathway and downregulated pro-inflammatory and profibrotic cytokines to modulate inflammatory and fibrotic responses. This indicates that dermaprazole is useful clinically for the prevention and/or treatment of at least radiation dermatitis; a common morbid complication that impacts a large number of patients across oncology.

Embodiments of the disclosure include methods of preventing or treating at least chemoradiation-induced inflammation and scarring (fibrosis); radiation-induced dermal inflammation and fibrosis; scleroderma; Mixed Connective Tissue Disease (MCTD); rheumatic diseases including Rheumatoid Arthritis (RA); lupus; polymyositis; dermatomyositis; atopic dermatitis; seborrheic dermatitis; Raynaud Disease; mucositis of any kind including oral or non-oral tissues; scarring of any kind or cause; keloids; chloracne; acne; wrinkled skin; aging skin; oxidative stress of skin; sunburn; photodamage; skin barrier protection; skin barrier photoprotection; skin cancer; skin grafts or transplanted skin; plastic or cosmetic surgery; cold injury and frostbite; psoriasis; vitiligo; allergic dermatitis; atopic dermatitis; inflammatory skin conditions of any kind or cause; wound healing; and/or aphthous ulcers. Such methods encompass delivering to an individual in need of prevention or treatment of at least chemoradiation-induced inflammation and scarring (fibrosis); radiation-induced dermal inflammation and fibrosis; scleroderma; Mixed Connective Tissue Disease (MCTD); rheumatic diseases including Rheumatoid Arthritis (RA); lupus; polymyositis; dermatomyositis; atopic dermatitis; seborrheic dermatitis; Raynaud Disease; oral mucositis; scarring of any kind or cause; keloids; chloracne; acne; wrinkled skin; aging skin; oxidative stress of skin; sunburn; photodamage; skin barrier protection; skin barrier photoprotection; skin cancer; psoriasis; vitiligo; allergic dermatitis; atopic dermatitis; inflammatory skin conditions of any kind or cause; wound healing; and/or aphthous ulcers an effective amount of one or more PPIs in any form. In specific embodiments, the PPIs are formulated in a topical formulation, such as a cream or gel, for example.

When more than one PPI is utilized for methods of preventing or treating one of the above-mentioned medical conditions, they may be administered to the individual at the same time or at different times. In either case, the multiple PPIs may be in the same or different compositions.

Embodiments of the disclosure include methods of improving the health and appearance of skin that will be and/or that has been exposed to an environmental condition that directly or indirectly affects the skin or dermis. As an example, there are methods of improving the health and appearance of skin or dermis that has been and/or will be exposed to one or more receptor tyrosine kinase inhibitors (imatinib, gefitinib and/or erlotinib, as examples), one or more monoclonal antibodies or conjugates thereof (blinatumomab, Bevacizumab, Cetuximab, alemtuzumab, trastuzumab, Ibritumomab tiuxetan, Brentuximab vedotin, Ado-trastuzumab emtansine, as examples) one or more immune checkpoint inhibitors (drugs that target PD-1 (Pembrolizumab, Nivolumab, Cemiplimab) or PD-L1 (Atezolizumab, Avelumab, Durvalumab), or CTLA-4 (Ipilimumab), as examples), radiation and/or chemoradiation for any purpose, including cancer treatment. The health and appearance of the skin may or may not include restoring the skin to its pre-therapy health and appearance. The health and/or appearance of the skin may or may not be judged based on the presence of inflammation, scarring, edema, blisters, necrosis, ulceration, atrophy, spider veins, thickening, itching, redness, or a combination thereof, for example.

Embodiments of the disclosure include methods of preventing or treating hair loss or hair thinning of any kind. In specific embodiments, the hair loss is as a result of having chemoradiation-induced inflammation and scarring (fibrosis); radiation-induced dermal inflammation and fibrosis; scleroderma; Mixed Connective Tissue Disease (MCTD); rheumatic diseases including Rheumatoid Arthritis (RA); lupus; polymyositis; dermatomyositis; atopic dermatitis; seborrheic dermatitis; and/or Raynaud Disease. The methods may reverse or prevent or delay hair loss or thinning as a result of having or being treated for chemoradiation-induced inflammation and scarring (fibrosis); radiation-induced dermal inflammation and fibrosis; scleroderma; Mixed Connective Tissue Disease (MCTD); rheumatic diseases including Rheumatoid Arthritis (RA); lupus; polymyositis; dermatomyositis; atopic dermatitis; seborrheic dermatitis; and/or Raynaud Disease. The hair loss or thinning associated with the chemoradiation-induced inflammation and scarring (fibrosis); radiation-induced dermal inflammation and fibrosis; scleroderma; Mixed Connective Tissue Disease (MCTD); rheumatic diseases including Rheumatoid Arthritis (RA); lupus; polymyositis; dermatomyositis; atopic dermatitis; seborrheic dermatitis; and/or Raynaud Disease may or may not be permanent. In at least certain cases, the method of preventing hair loss may prevent complete loss of hair or may prevent partial loss of hair, including delaying the onset of hair loss or reducing the amount of hair that is loss. When treating hair loss, the treatment may include causing hair that had been lost to re-grow, and part or all of the hair that had been lost may re-grow in methods of the disclosure.

In a specific embodiment, the methods include treatment or prevention of radiation-induced dermatitis. Radiation dermatitis is a dose-limiting normal tissue toxicity to the skin that occurs in a large proportion of cancer patients who receive radiation therapy. Radiation dermatitis manifests as an inflammatory reaction at the site of irradiation and may include redness, desquamation, loss of hair and necrotic changes. These clinical manifestations may also be accompanied by flaking, itching, pain, edema, blisters, skin retraction, induration, and infection. In moderate-to-severe cases, the inflammatory skin reaction may progress to fibrosis that permanently scars the irradiated tissue. This may result in significant compromise in the quality of life of the affected patients and may force the discontinuation of radiation therapy and threaten relapse of the underlying cancer (Spalek, 2016). Despite intensifying research effort, there is no effective therapy for radiation-induced dermatitis. There lacks effective prophylactic or therapeutic regimen that mitigates this commonly occurring complication of cancer therapy. Because of the central role of oxidative stress and inflammation in radiation-induced normal tissue toxicities, there are ongoing efforts to test and develop antioxidants and anti-inflammatory molecules to protect normal tissues from radiation toxicities including dermatitis, mucositis, pneumonitis, proctitis and esophagitis. In specific embodiments of the disclosure, one or more PPIs including at least dermaprazole, mitigates ionizing radiation-induced dermatitis.

Embodiments of the disclosure include the use of one, two, three, or more PPIs in combination to treat the inflammatory side effects of one or more cancer therapies, including one or more chemotherapeutic agents and/or radiation. In cases where an individual is treated that has been, is being, and/or will be treated with one or more chemotherapy drugs, the chemotherapy drug may be but is not limited to an anthracycline, such as doxorubicin (Adriamycin) and epirubicin (Ellence); a taxane, such as paclitaxel (Taxol) and docetaxel (Taxotere); 5-fluorouracil (5-FU); Cyclophosphamide (Cytoxan); and/or Carboplatin (Paraplatin), cisplatin (Platinol); Bortezomib (Velcade); Chlorambucil (Leukeran); Cyclophosphamide (Cytoxan, Neosar); Gemcitabine (Gemzar); Gleevec; Irinotecan (Camptosar); Irinotecan liposome injection (Onivyde); Methotrexate (Rheumatrex, Trexall); Oxaliplatin (Eloxatin); Trastuzumab (Herceptin); bleomycin (Blenoxane), etoposide (Etopophos), mitomycin (Mitosol), cetuximab (Erbitux), capecitabine (Xeloda), and a combination thereof.

Embodiments of the disclosure include methods of treating the skin or dermis having inflammation, scarring, edema, blisters, necrosis, ulceration, atrophy, spider veins, thickening, itching, redness, or a combination thereof, or preventing any one or more of these, irrespective of their cause(s) by providing a therapeutically effective amount of one or more PPIs to the individual.

Embodiments of the disclosure include methods of treatment or prevention of one or more dermatological medical conditions for which one or more PPIs is effective. The dermatological condition may have one or more of the following symptoms: inflammation, scarring, edema, blisters, necrosis, ulceration, atrophy, spider veins, thickening, itching, redness, or a combination thereof.

In cases wherein the individual has or is susceptible to dermatitis, the dermatitis may be atopic dermatitis (eczema), seborrheic dermatitis, or contact dermatitis, for example.

In some embodiments, the individual in need of one or more PPIs for indications encompassed herein have been given, are being given, and/or will be given an effective amount of another therapeutic or other agent, such as one or more corticosteroids, one or more antibiotics, zinc, Amifostine, Silver leaf nylon dressing, one or more pain relievers, (such as lidocaine, narcotics, non-steroidal anti-inflammatory drugs, etc.) or a combination thereof.

In particular embodiments, one or more PPIs are provided to an individual in a therapeutically effective amount. In situations where more than one PPI is to be administered to an individual, they may be administered at the same time or at different times. When administered at the same time, they may or may not be formulated in the same composition. When they are administered at different times, the span of time between the administrations may be within a minute, within 1-59 minutes, within 1-24 hours, within 1-7 days, within 1-4 weeks, within 1-12 months, and so forth, of each other.

In specific embodiments, the PPIs are provided to an individual in need of treatment or prevention of inflammation and/or fibrosis related to cancer therapy and are not provided to the individual for the treatment of the cancer itself. The individual may be determined to be in need of therapy for treatment or prevention of inflammation and/or fibrosis including that is related to cancer therapy. The PPIs may be formulated for use related to treatment or prevention of a condition other than for cancer treatment. In some embodiments, however, encompassed herein are topical PPI(s) being used to relieve inflammation and/or fibrosis related to cancer that also sensitizes the cancer being treated to cancer therapy, including at least chemoradiation therapy. In particular embodiments, the PPIs are utilized to treat skin cancer or any medical condition listed herein, such as topically.

II. Examples of PPIs and Compositions Thereof

PPIs are normally utilized for gastric conditions, but this disclosure encompasses reformulation of their use for conditions other than gastric conditions to formulations useful for one or more dermatological conditions of any kind, including at least chemotherapy- and/or radiation therapy-induced tissue inflammation and scarring. This characteristic of PPIs is devoid of the indication (i.e., treatment of gastritis) for which they are FDA-approved. PPIs directly regulate many of the inflammatory cytokines generated in response to many of the commonly used chemotherapeutic agents or ionizing radiation, as encompassed herein. Many of these esomeprazole-regulated cytokines are reported to be upregulated in dermatitis, for example.

Embodiments of the disclosure include one or more PPIs for treatment of one or more dermatological conditions of any kind. The PPIs may be formulated into any kind of composition suitable for the treatment or prevention required. The PPIs may be formulated for local or systemic administration, although in particular embodiments the administration is not for a gastric application. When more than one PPI is provided to an individual, they may or may not be formulated in the same composition.

As a specific example, the inventors generated topical esomeprazole cream for direct application to the skin to prevent and/or treat chemoradiation-induced dermatitis, and they demonstrate control of inflammation in vitro and in an animal model. In accordance, the PPIs can be reformulated in various preparations including cream (e.g. for dermatitis), liquid (e.g. for enteritis), troche (e.g. for oral mucositis), and suppository (e.g. for proctitis) to prevent and/or treat chemoradiation-induced tissue inflammation and scarring, as examples.

In particular embodiments, the PPI is Esomeprazole, Omeprazole, Lansoprazole, Dexlansoprazole, Pantoprazole, Rabeprazole, Ilaprazole, or a combination thereof. When an individual is provided multiple PPIs, they may or may not be formulated in different types of formulations, regardless of the regimen provided to the individual.

In particular embodiments, the one or more PPIs are formulated in a composition with one or more other agents for a therapeutic or other purpose. Examples include one or more chemotherapeutics, one or more corticosteroids, one or more antibiotics, zinc, Amifostine, Silver leaf nylon dressing, one or more pain relievers, or a combination thereof. One particular embodiment of a composition comprises one or more PPIs with lidocaine that for example may be used for oral mucositis.

Pharmaceutical compositions of the present disclosure comprise an effective amount of one or more PPIs dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one PPI or additional active ingredient will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The one or more PPIs may comprise different types of carriers depending on whether it is to be administered in solid, liquid or aerosol form, and whether it need to be sterile for such routes of administration as injection. The present invention can be administered topically, locally, intradermally, transdermally, subcutaneously, mucosally, in creams or gels, and so forth, although in certain cases the PPIs are administered intravenously, intrathecally, intraarterially, intraperitoneally, sublingually, intranasally, intravaginally, intrarectally, intramuscularly, orally, by inhalation (e.g., aerosol inhalation), injection (intraarticular, subcutaneous, etc.), as eye drops, infusion, continuous infusion, localized perfusion bathing target cells directly, via a catheter, via a lavage, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The one or more PPIs may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, magnesium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present disclosure suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants, including but not limited to sodium metabisulfite, glutathione or N-acetyl cysteine, to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition may be combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, e.g., denaturation in the stomach. Examples of stabilizers for use in the composition include buffers, amino acids such as glycine and lysine, carbohydrates such as dextrose, mannose, galactose, fructose, lactose, sucrose, maltose, sorbitol, mannitol, etc.

In further embodiments, the present disclosure may encompass the use of a pharmaceutical lipid vehicle compositions that include one or more PPIs, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the PPI(s) may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an individual can be determined by physical and physiological factors such as body weight, severity of condition, the type of disease being treated, previous or concurrent therapeutic interventions, and/or on the route of administration. Depending upon the dosage and the route of administration, the number of administrations of a preferred dosage and/or an effective amount may vary according to the response of the individual. The practitioner responsible for administration will, in any event, determine the concentration of active ingredient(s) in a composition and appropriate dose(s) for the individual.

In certain embodiments, pharmaceutical compositions may comprise, for example, at least about 0.1% of an active compound. In other embodiments, the active compound may comprise between about 1% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 milligram/kg/body weight or more per administration, and any range derivable therein. In non-limiting examples of a derivable range from the numbers listed herein, a range of about 5 milligram/kg/body weight to about 100 milligram/kg/body weight, about 5 microgram/kg/body weight to about 500 milligram/kg/body weight, etc., can be administered, based on the numbers described above.

In certain embodiments, the PPI is formulated in a particular concentration in a composition. In specific embodiments, the PPI is formulated in a range of 1%-100% w/w. In specific cases, the PPI is formulated in a range of 1%400%, 1%-75%, 1%-50%, 1%-25%, 10%-100%, 10%-75%, 10%-50%, 25%-100%, 25%-75%, 25%-50%, 1%-20%, 1%-18%, 1%-16%, 1%-15%, 1%-12%, 1%-10%, 1%-8%, 1%-6%, 1%-5%, 1%-4%, 1%-3%, 1%-2%, 2%-5%, 2%-4%, 2%-3%, 3%-5%, 3%-4%, 4%-5%, 5%-20%, 5%-15%, 5%-10%, 10%-20%, 10%-15%, 12%-15%, 12%-20% w/w. The PPI may be formulated in a concentration of 1%, 2%, 3%, 4%, or 5% w/w, in some cases.

In particular embodiments, the concentration of proton pump inhibitor is not greater than 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21 20%, 19, 18, 17, 16, 15, 14, 13, 12, 11, 10, 9, 8, 7, 6, 5, 4, 3, 2, or 1% w/w.

A. Parenteral Compositions and Formulations

In further embodiments, one or more PPIs may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

B. Alimentary Compositions and Formulations

Although the PPI(s) are preferably formulated for parenteral administration, in an alternative embodiment of the present disclosure, the one or more PPIs may be formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration, the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

C. Miscellaneous Pharmaceutical Compositions and Formulations

In other preferred embodiments of the invention, the active compound PPI(s) may be formulated for administration via various miscellaneous routes, for example, topical (i.e., transdermal with or without the aid of patches or bandages) administration, mucosal administration (intranasal, vaginal, etc.) and/or inhalation.

Pharmaceutical compositions for topical administration may include the active compound formulated for a medicated application such as an ointment, paste, cream, gel, or powder. Ointments include all oleaginous, adsorption, emulsion and water-solubility based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, eye suspensions, eye gels, eye ointments, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

III. Kits of the Disclosure

Any of the PPI compositions described herein may be comprised in a kit. In a non-limiting example, one or more PPIs may be comprised in a kit. The kits will thus comprise, in suitable container means, a PPI(s) and a lipid, and/or an additional agent of the present disclosure. The one or more PPI compositions may be formulated as a liquid, troche, suppository, cream, solid, tablet, pill, aerosol, gel, film, foam, ointment, paste, cream, gel, powder, or combination thereof, for example. In specific embodiments, one or more PPI compositions formulated for use as prevention or treatment of indications encompassed herein and also may be provided in a kit with one or more cancer drugs, one or more corticosteroids, one or more antibiotics, zinc, Amifostine, Silver leaf nylon dressing, one or more pain relievers, or a combination thereof. In some cases, a first PPI is packaged with a first chemotherapy drug or other therapeutic while a second packaging comprises a second PPI packaged with a second chemotherapy drug or other therapeutic. In such cases when a PPI and a chemotherapy drug and optionally another therapeutic are packaged in the same package they may be housed within different containers within the package.

The kits may comprise a suitably aliquoted PPIs, lipid and/or additional agent compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container or compartment into which the additional components may be separately placed. However, various combinations of components may be comprised in a vial. The kits of the present disclosure also will typically include a means for containing the one or more PPIs, lipid, additional agent, and any other reagent containers in close confinement for commercial sale. Such containers may include injection or blow-molded plastic containers into which the desired vials are retained.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means or compartment in which the formulation(s) are placed, preferably, suitably allocated. The kits may also comprise a second container means for containing a sterile, pharmaceutically acceptable buffer and/or other diluent.

The kits of the present disclosure will also typically include a means for containing the vials in close confinement for commercial sale, such as, e.g., injection and/or blow-molded plastic containers into which the desired vials are retained.

### EXAMPLES

The following examples are included to demonstrate particular embodiments of the disclosure. It should be appreciated by those of skill in the art that the techniques disclosed in the examples that follow represent techniques discovered by the inventor to function well in the practice of the disclosure, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the disclosure.

### Example 1

### Proton Pump Inhibitors and Methods of Use in Chemoradiation-Induced Tissue Inflammation and Scarring

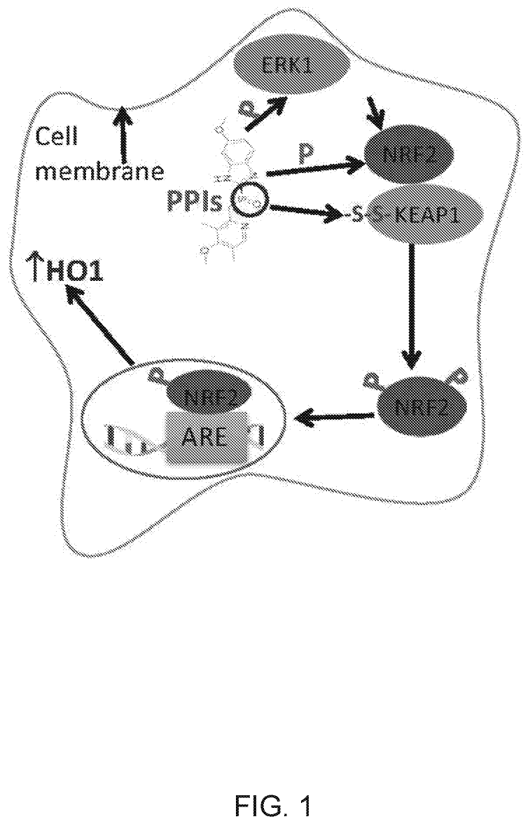

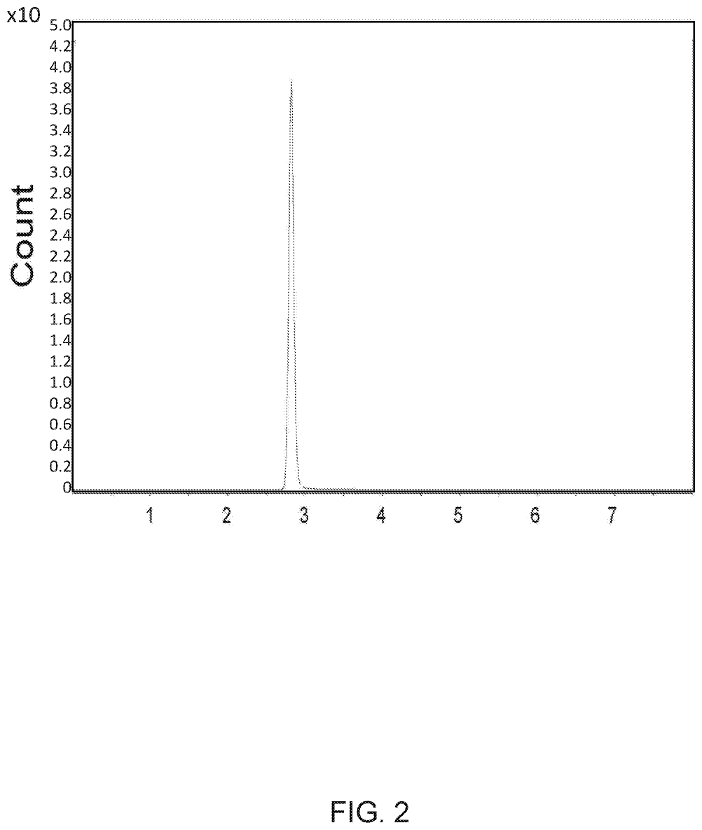

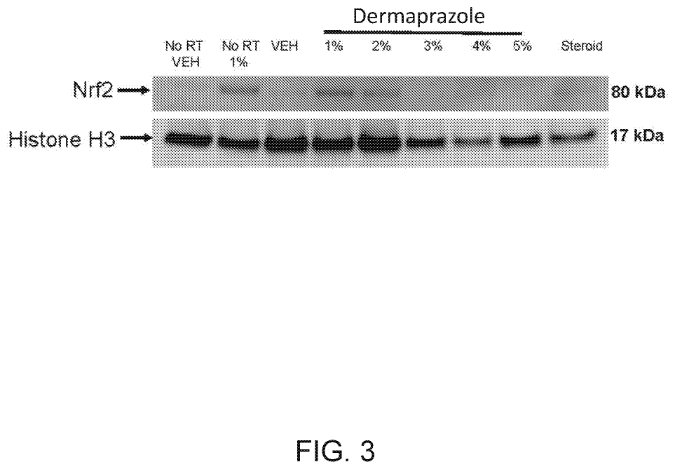

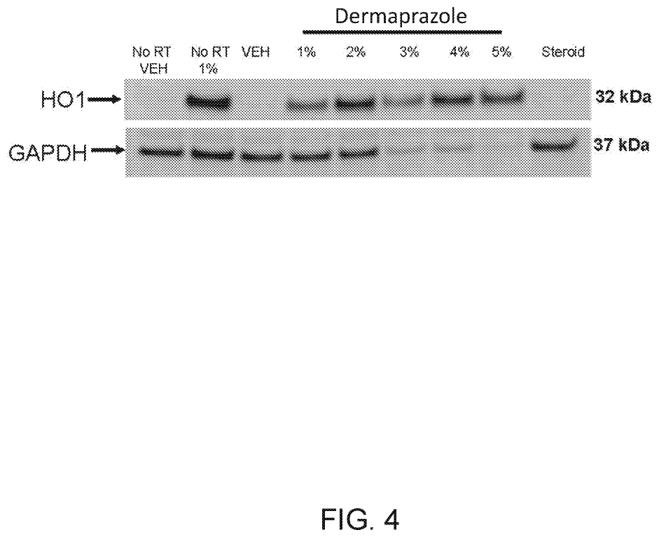

PPIs activate HO1 by inducing nuclear translocation of Nrf2 either through phosphorylation of ERK1, Nrf2 itself and/or attacking the sulfhydryl group in Keap1 and disassociating Keap1-Nrf2 complex (FIG. 1). As merely an example, a cream of esomeprazole was prepared and termed dermaprazole. FIG. 2 shows a representative chromatography (LC-MS) data showing stability of dermaprazole after 30 days of formulation and storage. The single peak shows intact (i.e. degradation-free) dermaprazole. To show Nrf2 levels, FIG. 3 provides protein expression data showing induction of Nrf2 by dermaprazole (1-2%) in nuclear fraction of cell extracts from human 3D skin model at baseline (“No RT”) and after 14 gray irradiation. Protein levels of histone H3 are shown as loading control, and anti-Nrf2 Rabbit monoclonal Antibody (Abcam; ab62352,1:250) and Rabbit polyclonal to Histone H3 (Abcam; ab1791; 1:3000) are used. FIG. 4 demonstrates protein expression data showing induction of HO1 by dermaprazole (1-5%) in total cell extracts from human 3D skin model at baseline (“No RT”) and after 14 gray irradiation. GAPDH protein levels were used as a loading control.

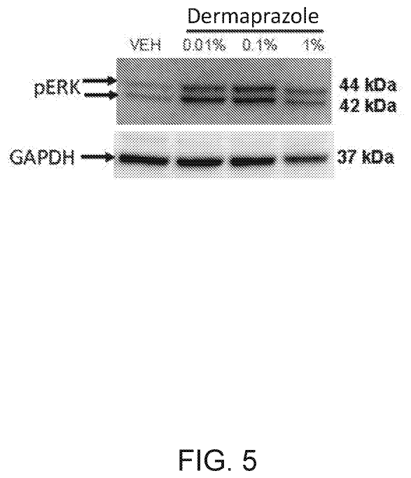

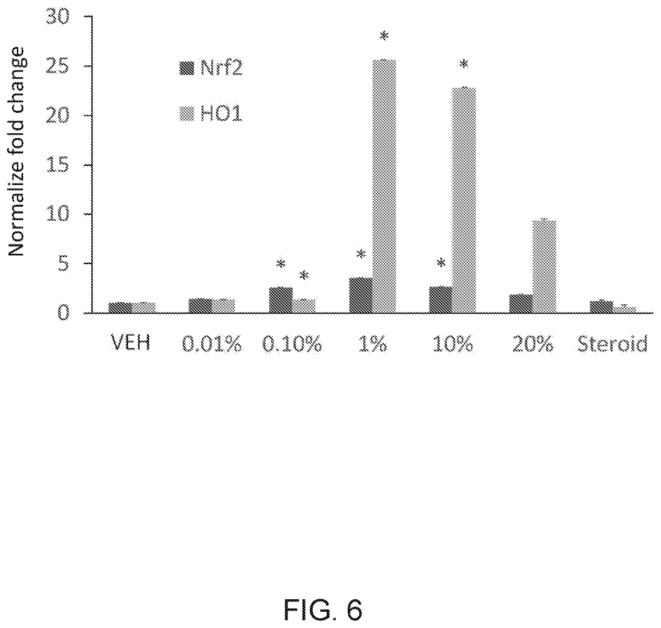

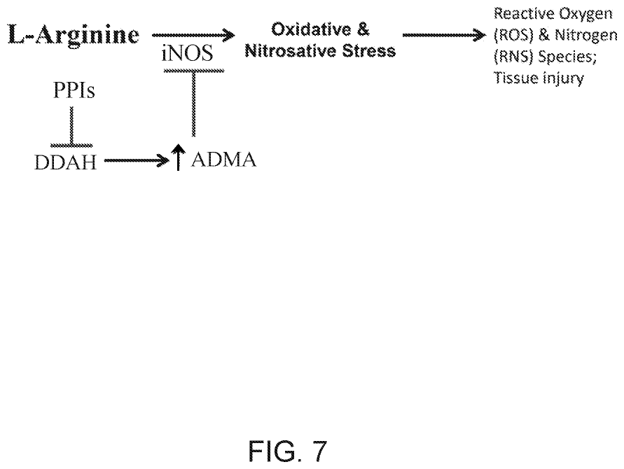

Protein expression data demonstrating activation/phosphorylation of ERK1/2 (pERK1/2) by dermaprazole in irradiated (14 gray) human 3D skin tissue is shown in FIG. 5, with GAPDH protein levels as a loading control. Gene expression data demonstrating induction of Nrf2 and HO1 by dermaprazole in a human 3D skin model (EpiDermFT; MatTekCorporation) following 14 gray irradiation is provided in FIG. 6.

### Example 2

### Topical Esomeprazole Mitigates Radiation-Induced Dermal Inflammation and Fibrosis

Dermaprazole is a homogenous product with consistency in appearance, color and odor: The inventors' study of the grittiness, color and odor of dermaprazole over time shows that the product has homogenous appearance with relatively stable odor and light brown color at low drug strength (0.01% to 2%) that increased in intensity towards dark brown over concentration, temperature and time. The increased intensity in the color of the product is in part due to oxidation because reducing the storage condition from room temperature to 4° C., keeping the container tightly closed or adding antioxidant (0.1% sodium metabisulfite) helped maintain the original color of the product (data not shown). However, it was consistently observed that dermaprazole containing higher drug concentration (>5% w/w) was relatively less stable than dermaprazole containing lower concentration of active drug (0.01% to 4% w/w) with the highest concentrations (5% to 20%) showing rapid change in color towards dark brown or purple upon storage at room temperature or exposure to room air. This apparent lack of chemical stability was consistent with lack of biological activity including failure to modulate the expression of target genes. These chemical and biological properties of dermaprazole urged us to focus most of our downstream work on lower drug strengths (1 to 2%) that are found to be more stable and reproducible in engaging biological targets.

Physicochemical stability of dermaprazole: A LC-MS study showed that the concentration of esomeprazole molecules recovered from the cream was proportional to the strength of dermaprazole formulation, with the lowest strength of dermaprazole (i.e. 0.01%) correlating to the lowest recovery of esomeprazole molecules (Table 1).

TABLE 1
Measurement of esomeprazole concentration from dermaprazole cream by
LC-MS. The average esomeprazole concentration recovered from dermaprazole
cream was calculated from a standard curve constructed using esomeprazole
powder. Data shown is from duplicate experiments (Mean ± STD).

| | Drug Content (ng/mg cream, % recovery in reference to D 1) | | |
|---|---|---|---|
| Sample | Day 1 | Day 18 | Day 32 |
| Vehicle | 0 ± 0 | 0 ± 0 | 0 ± 0 |
| | (—) | (—) | (—) |
| 0.01% Dermaprazole | 11.2 ± 1.1 | 3.1 ± 0.3 | 1.7 ± 0.1 |
| | (100%) | (28%) | (15%) |
| 0.1% Dermaprazole | 94.5 ± 8.9 | 4.1 ± 0.4 | 3.6 ± 0.1 |
| | (100%) | (4%) | (4%) |
| 1% Dermaprazole | 24,584.1 ± 0.6 | 21,936.3 ± 482.8 | 17,872.8 ± 118.9 |
| | (100%) | (89%) | (73%) |
| 10% Dermaprazole | 231,650.0 ± 17156.4 | 207,320.0 ± 13375.2 | 152,570.4 ± 6220.8 |
| | (100%) | (89%) | (66%) |
| 20% Dermaprazole | 453,765.6 ± 13351.9 | 442,588.4 ± 3060.3 | 400,846.7 ± 1481.2 |
| | (100%) | (98%) | (88%) |

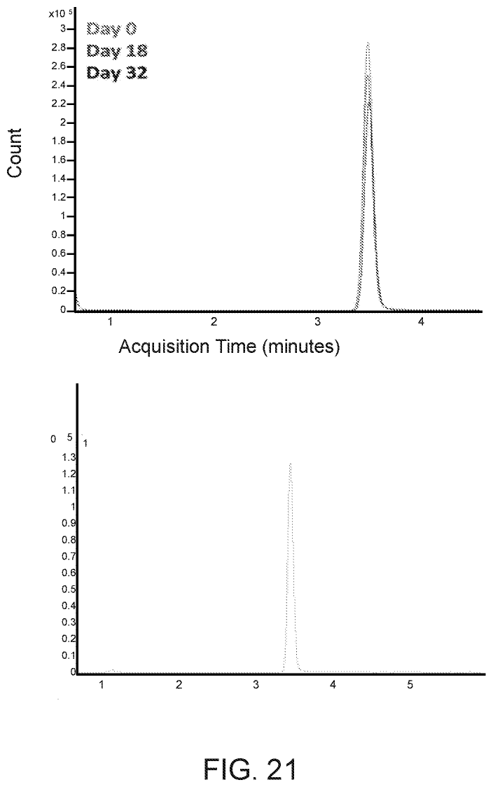

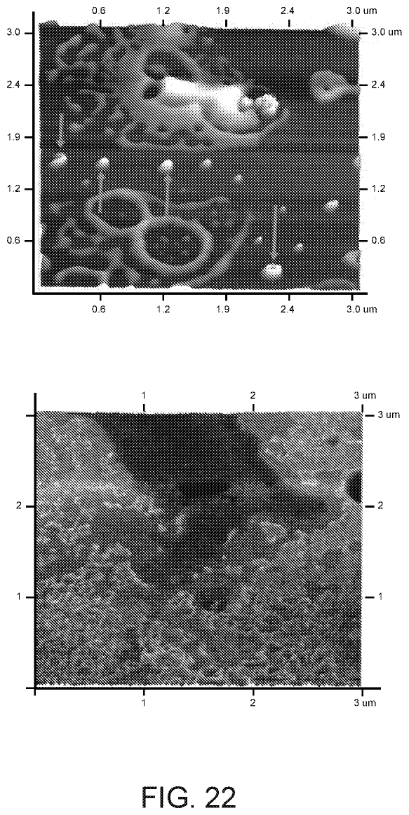

As expected, the vehicle control showed no presence of esomeprazole molecules. In addition, the chromatogram data showed that the signature of the peak intensity and acquisition time for esomeprazole in the cream after 1 month of storage (day 32) correlated well with that of freshly prepared dermaprazole cream (day 0) and esomeprazole powder that was not formulated into a cream (FIG. 21). Meanwhile, the atomic force microscopy scanning data showed that dermaprazole blended well into the cream base that was used as a carrier and formed a relatively stiffer and less adhesive micelle-like product compared to the cream base alone (FIG. 22).

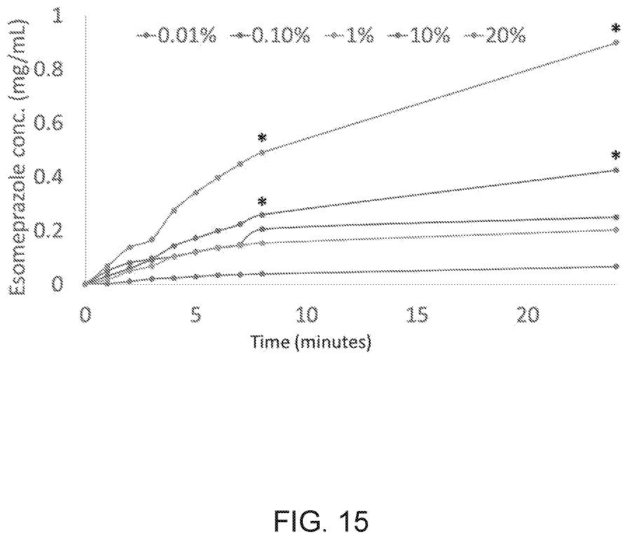

Retention and permeability of dermaprazole: Our drug permeation study showed that the various strengths of dermaprazole maintained variable skin retention properties, as well as permeability through dermal membrane over a course of time. More specifically, measurement of drug concentration in the receptor compartments of the Franz Diffusion Cell demonstrated that the lowest dermaprazole strengths (0.01% to 1%) do not show appreciable increase in the concentration of drug in the receptor chamber over time relative to baseline while the highest dermaprazole strengths (10% to 20%) showed significant increase in the amount of drug released over time (FIG. 15).

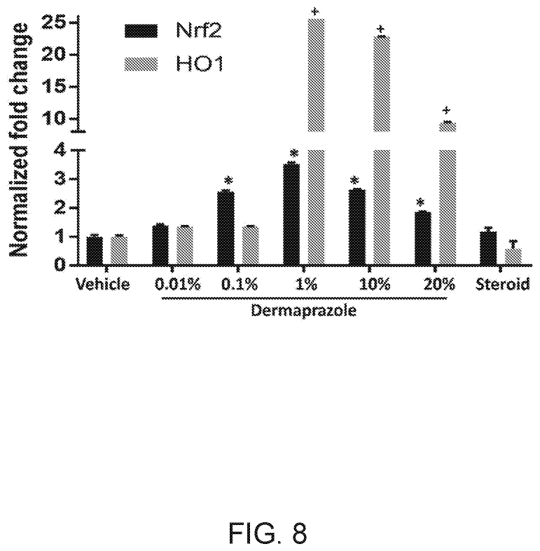

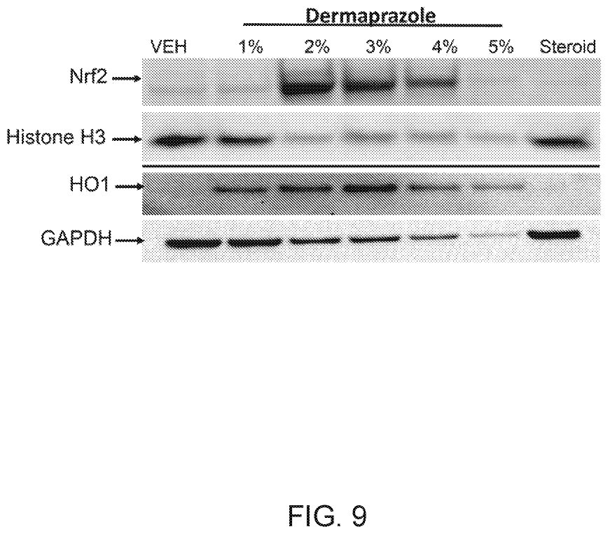

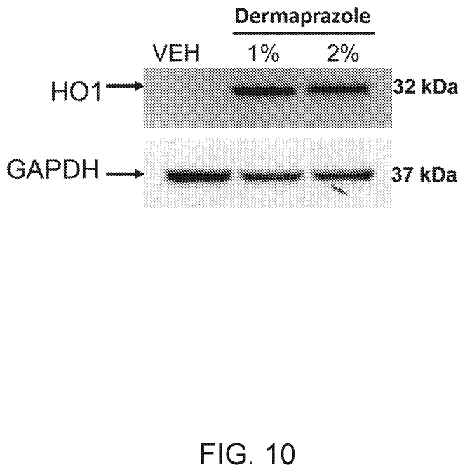

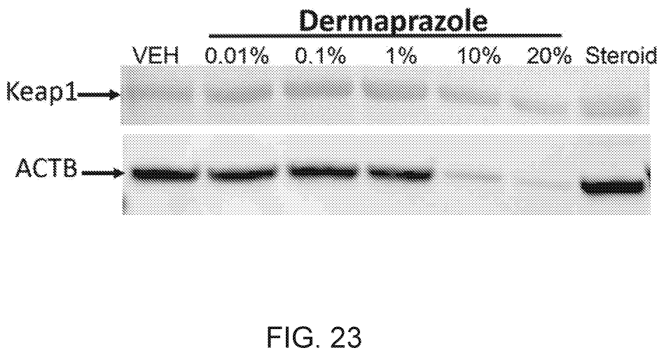

Induction of Nrf2-HO1 pathway by dermaprazole in 3D human skin model: Topical application of dermaprazole for 24 hours onto ionizing radiation-exposed 3D full-thickness human skin resulted in robust induction of the gene (FIG. 8) and protein (FIG. 9) expression of Nrf2 and HO1 in the dermal layer of the tissue. Our subcellular fractionation study also shows that Nrf2, which is physiologically compartmentalized in the cytoplasm of cells, is translocated into the nuclei (FIG. 9; top panel). Although one of the possible mechanisms for the nuclear translocation of Nrf2 is inhibition of Keap1 by dermaprazole, there was no detectable change in Keap1 expression upon dermaprazole treatment (FIG. 23). Intriguingly, the expression of HO1 was significantly induced in unirradiated human skin (FIG. 10) suggesting that the major antioxidant enzyme HO1 can be upregulated by dermaprazole despite exposure to radiation and oxidative stress response.

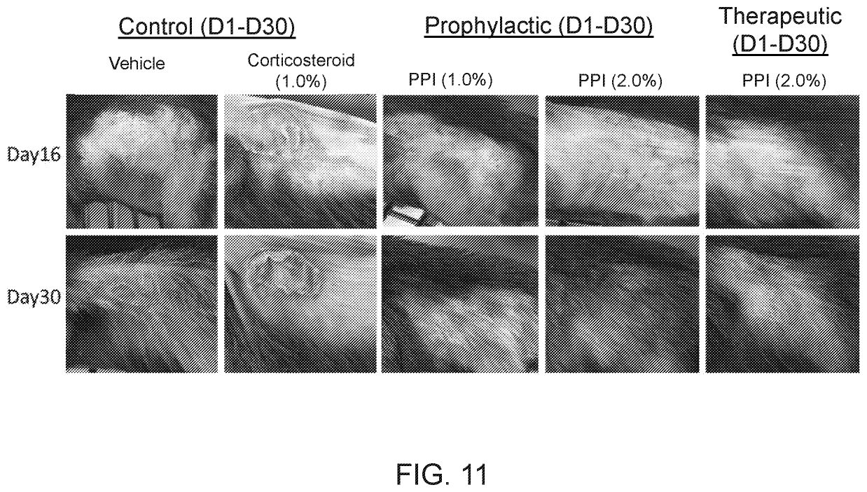

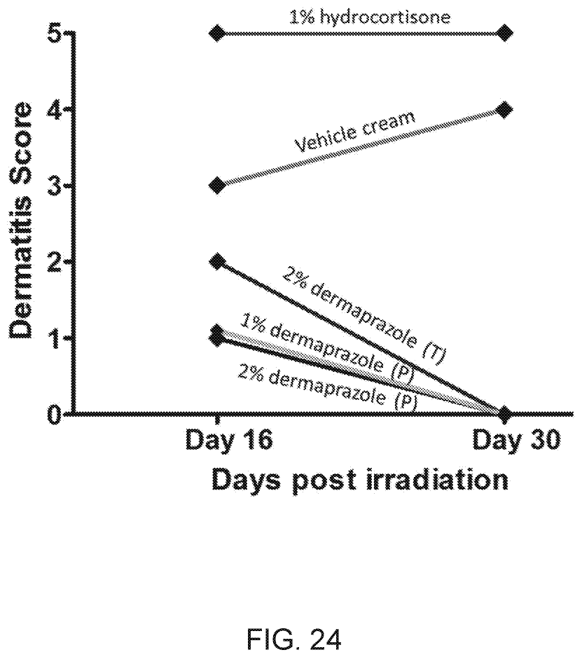

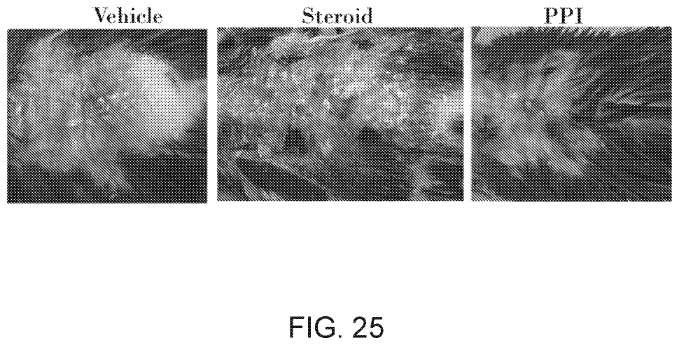

Efficacy of dermaprazole in suppressing radiation-induced dermatitis: The digital photography data from the mouse model of radiation-induced dermatitis revealed that treatment of animals with dermaprazole has substantial effect in improving the macroscopic appearance of the irradiated skin by day 16 and resulted in complete or nearly complete healing of the wound at the irradiation site in over 60% of the animals in either of the prophylactic dermaprazole groups by the end of the study at day 30 (FIG. 11). Intriguingly, therapeutic administration of dermaprazole after exposure to the full dose of ionizing radiation also resulted in significant closure of the wounds, and skin appearance that is comparable to that of animals that are treated with dermaprazole in a prophylactic course (FIG. 11). Meanwhile, moisturizing the irradiation site with vehicle cream also improved the appearance of the skin. However, treatment with 1% hydrocortisone had no effect on healing of the wounds in 90% of the animals in this model and, as a result, the animals in the corticosteroid group had severe skin necrosis at both 16 days and 30 days post-irradiation (FIG. 11). A board-certified dermatologist who was unaware of the treatment groups has evaluated and scored the degree of dermatitis using CTCAE criteria (version 4.0). The assessment shows that treatment with dermaprazole, whether administered prophylactically or therapeutically, has significantly reduced the degree of dermatitis by day 16 and normalized the appearance of the skin by day 30 (FIG. 24). The scleroderma model shows similar results (FIG. 25).

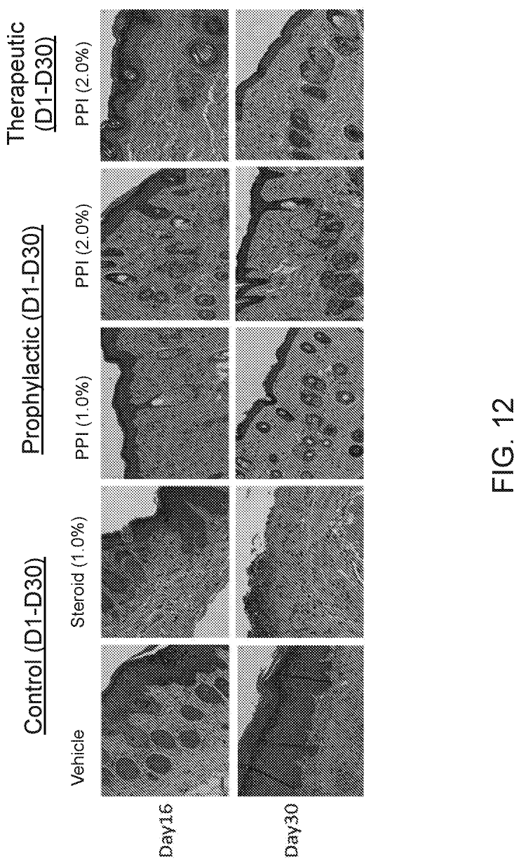

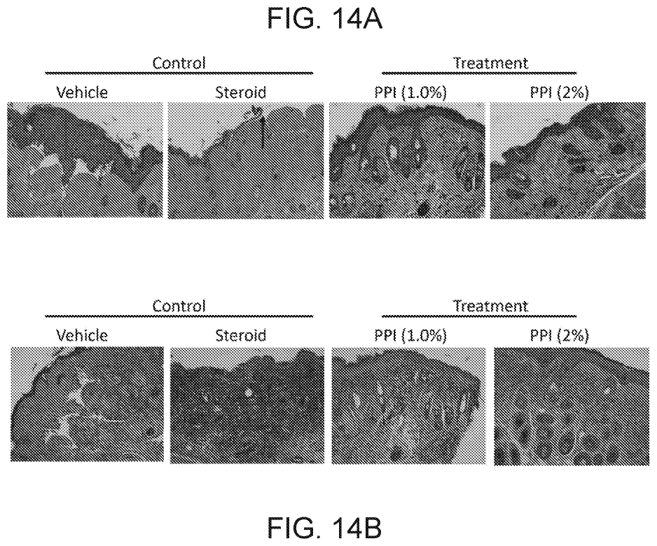

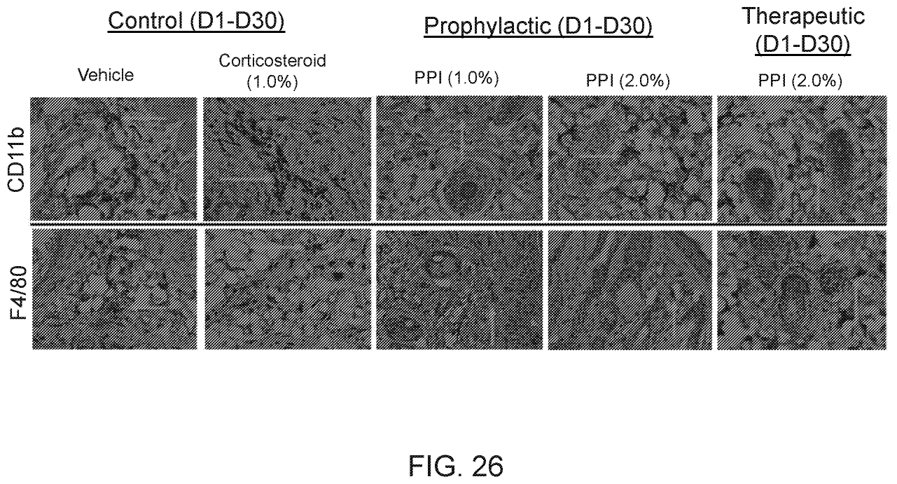

Dermaprazole reduces dermal inflammation: histological & genetic evidence: Consistent with the macroscopic improvement in skin appearance upon dermaprazole treatment, H&E staining of the skin tissue obtained from the irradiation site confirmed that prophylactic or therapeutic treatment with dermaprazole reduced epidermal thickening at day 16 and day 30 (FIG. 12). In addition, the histological scores of ulceration, necrosis, parakeratosis/crust, and overall inflammation were significantly reduced by dermaprazole treatment. The calculated mean score for each of these parameters showed that 1% dermaprazole administered in a prophylactic course reduced ulceration by 15%, necrosis by 18%, parakeratosis/crust by 2-fold, inflammation by 2-fold, and epidermal thickening by about 6-fold at day 16 in comparison to the steroid control. Encouragingly, the trend continued throughout the treatment period with reductions at day 30 by 2-fold, 5-fold, 88%, 86% and 86% respectively (FIG. S2). Similarly, 2% prophylactic dermaprazole reduced these scores by 1.5-fold, 6.5-fold, 88%, 1.3-fold and 100% respectively in comparison to the steroid control at day 30 while the 2% therapeutic dermaprazole reduced these histological scores by 1.3-fold, 4-fold, 70%, 100% and 80% respectively. In addition, immunohistochemical staining of paraffin-embedded skin tissues for the pan-leukocyte marker CD11b and the macrophage specific marker F4/80 showed that dermaprazole reduced recruitment of these cells into the injury site (FIG. 26). Intriguingly, dermaprazole showed similar efficacy in the scleroderma model (FIGS. 14A and 14B).

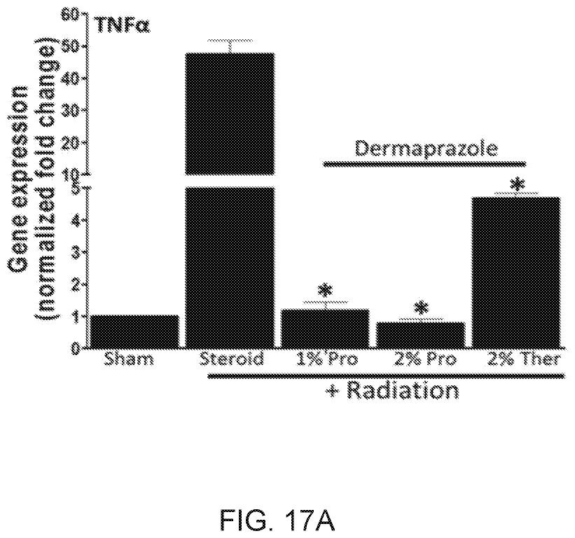

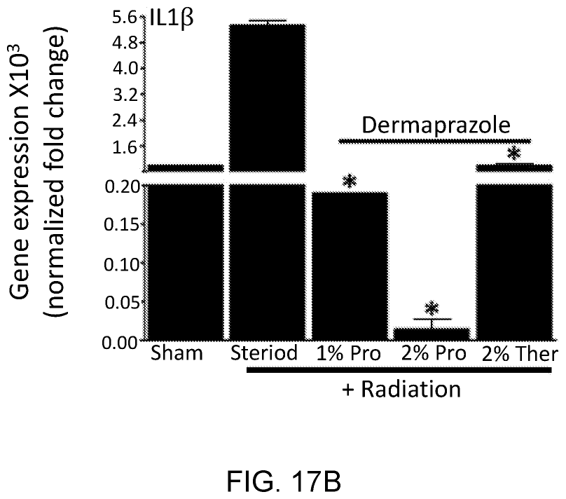

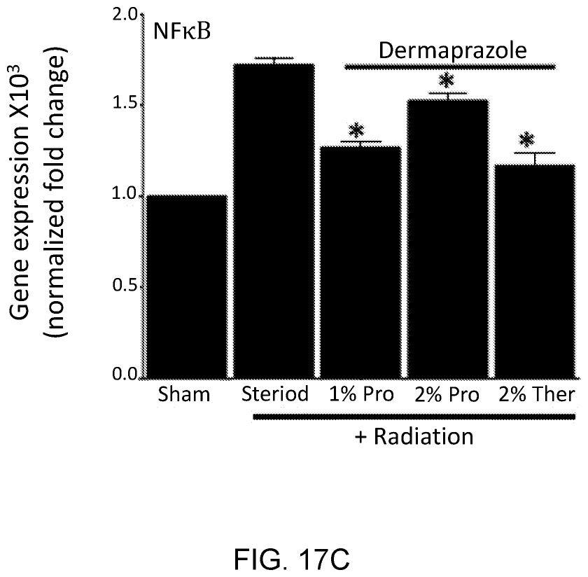

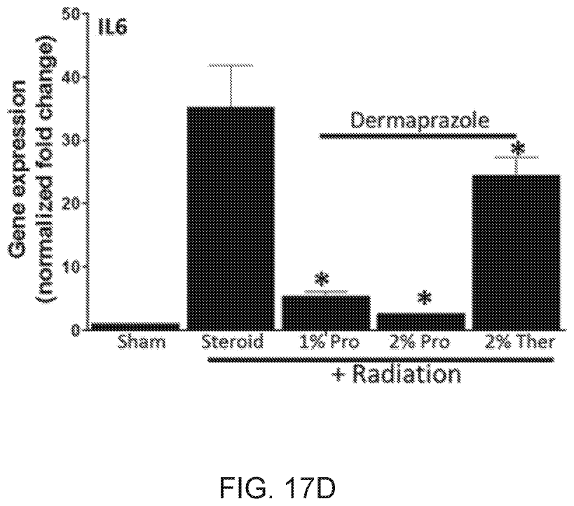

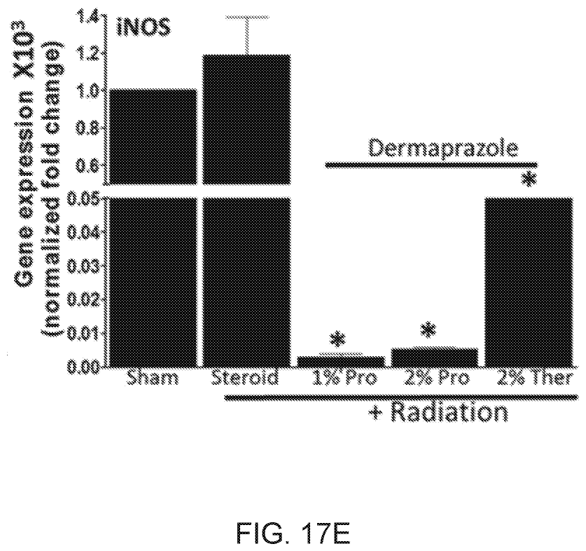

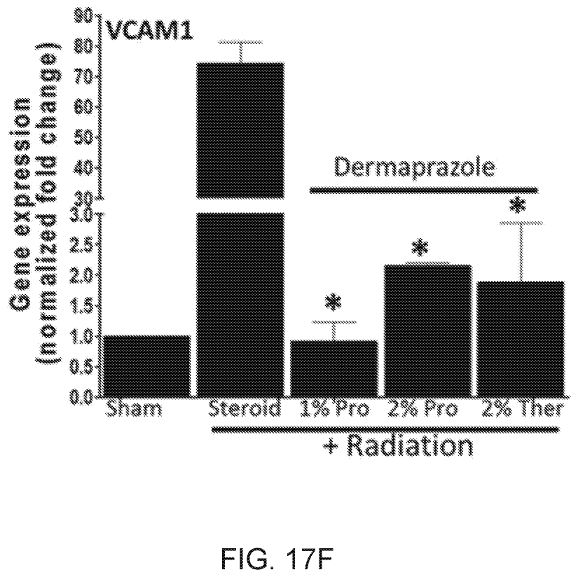

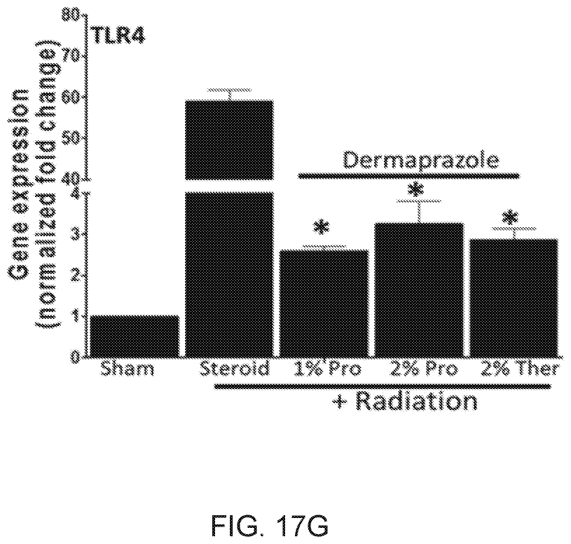

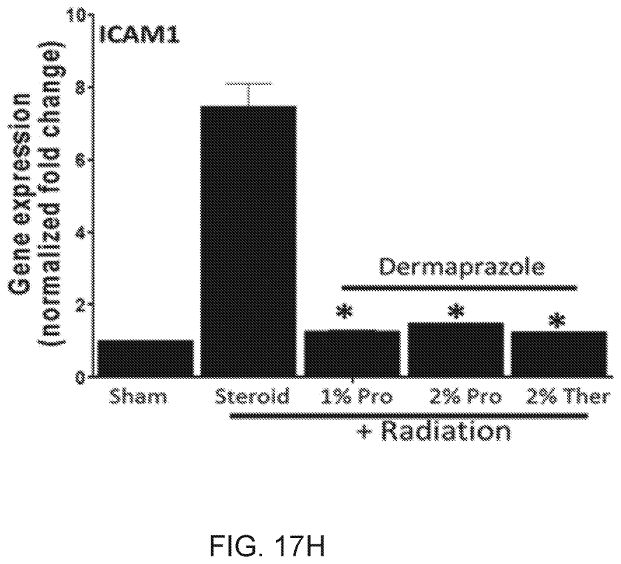

In addition, gene expression study probing markers of inflammation confirmed that dermaprazole treatment significantly downregulated the mRNA expression of many of the classic pro-inflammatory cytokines including TNFα, IL1β, NFkB, TLR4, IL6, ICAM1, VCAM1 and iNOS (FIGS. 17A-17H).

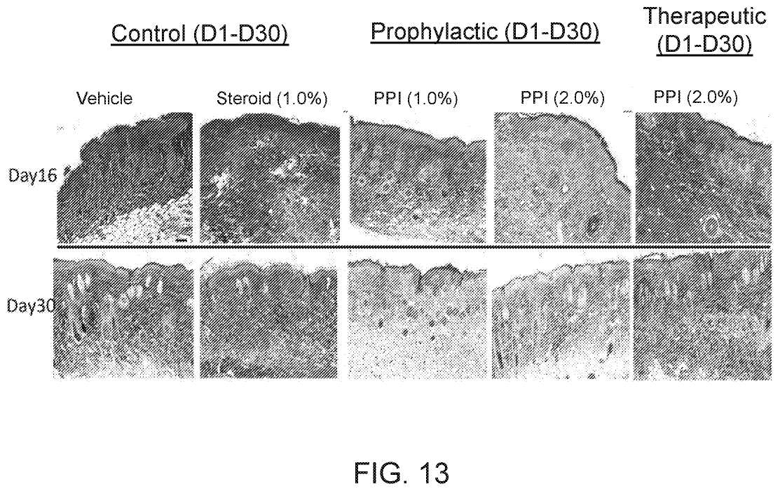

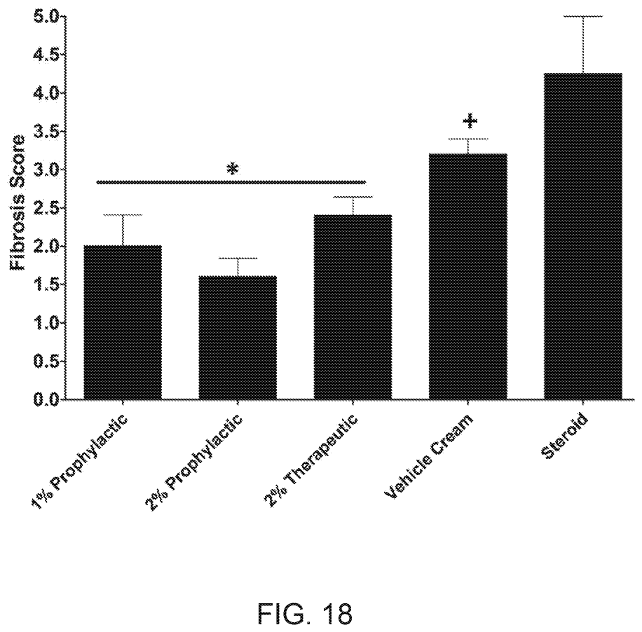

Dermaprazole reduces dermal fibrosis: immunohistochemical & genetic evidence: Masson's Trichrome stain of collagen in skin tissue explanted from the radiation dermatitis model showed that treatment with dermaprazole notably reduced collagen accumulation on days 16 and 30 in comparison to vehicle or corticosteroid controls (FIG. 13) indicating greater inhibition of fibrotic changes upon dermaprazole treatment. The trichrome stain scores indicated that there was significantly lower degree of fibrotic changes in the dermaprazole treatment groups (FIG. 18). Similarly, the degree of dermal fibrosis was reduced in the scleroderma model (FIG. 14B).

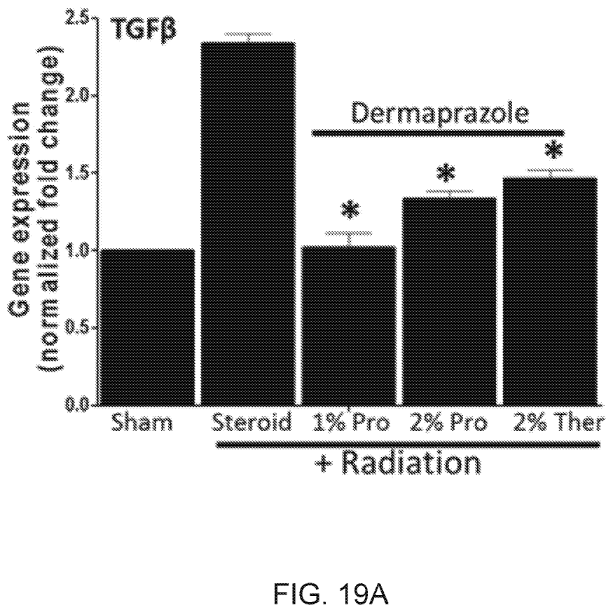

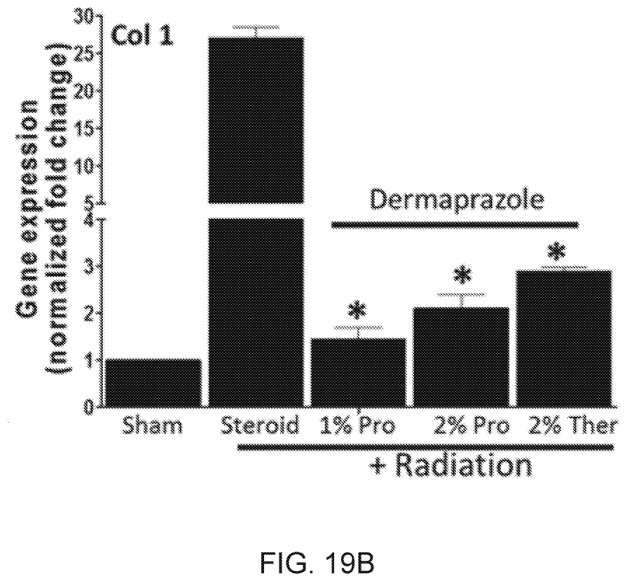

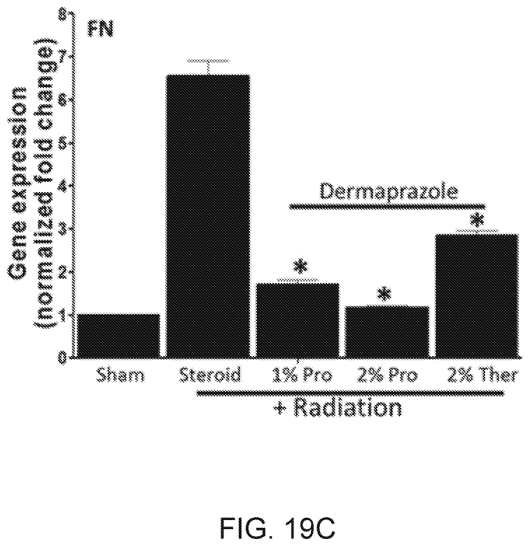

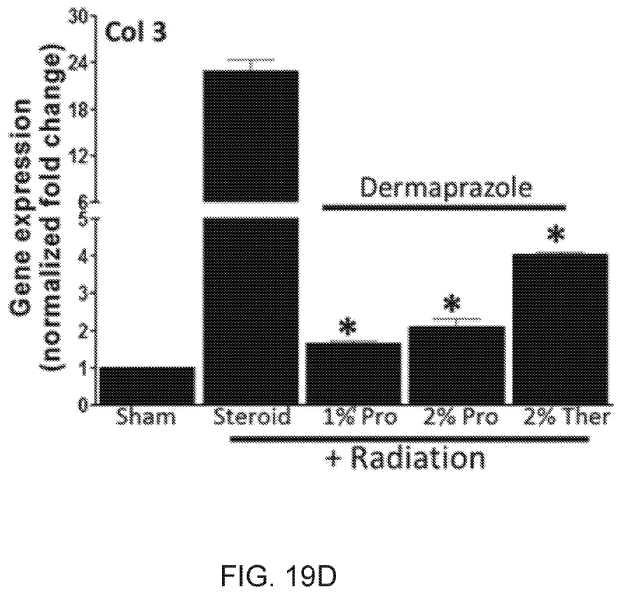

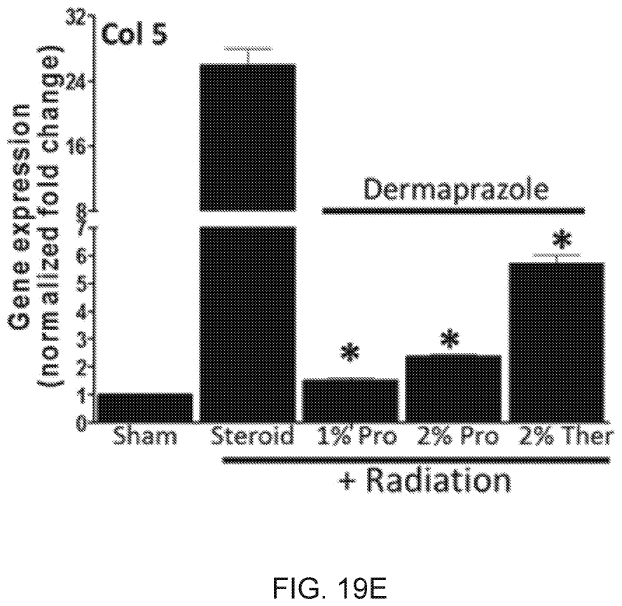

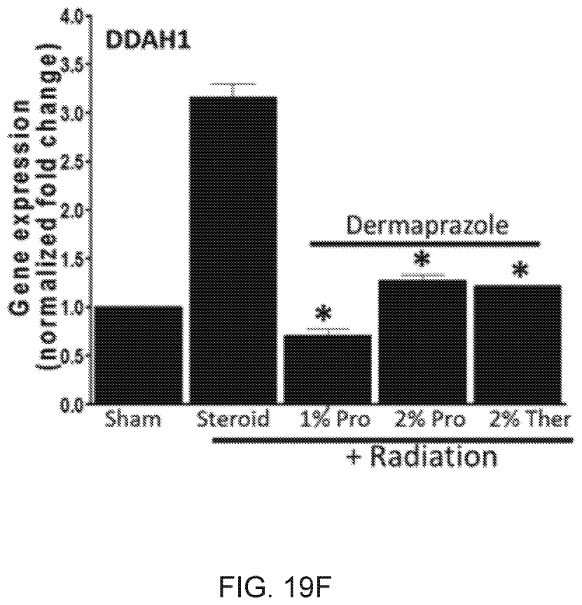

Gene expression study for profibrotic markers also confirmed that dermaprazole significantly downregulated the expression of TGFβ, DDAH1, collagen 1, 3, 5 and fibronectin (FIGS. 19A-19F).

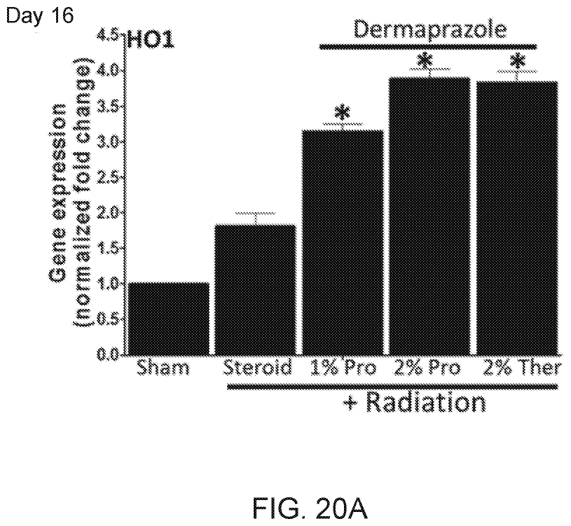

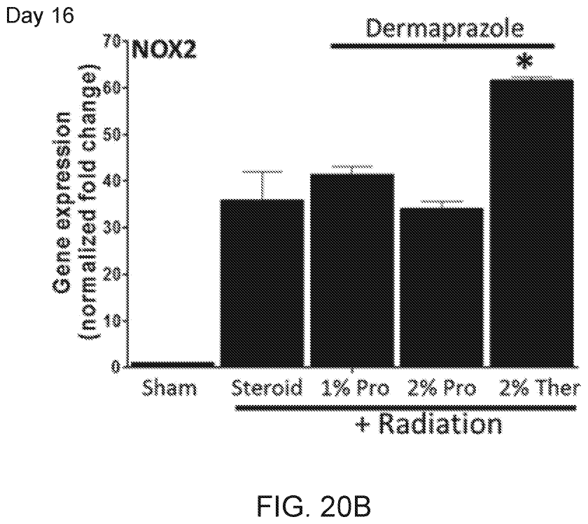

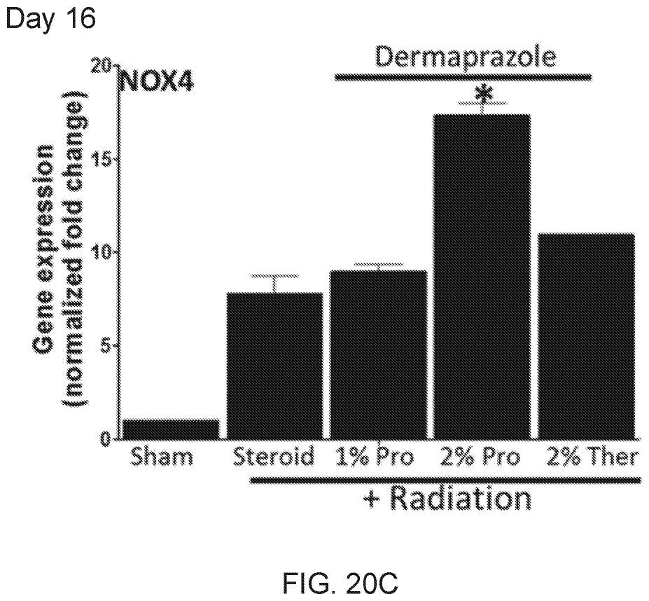

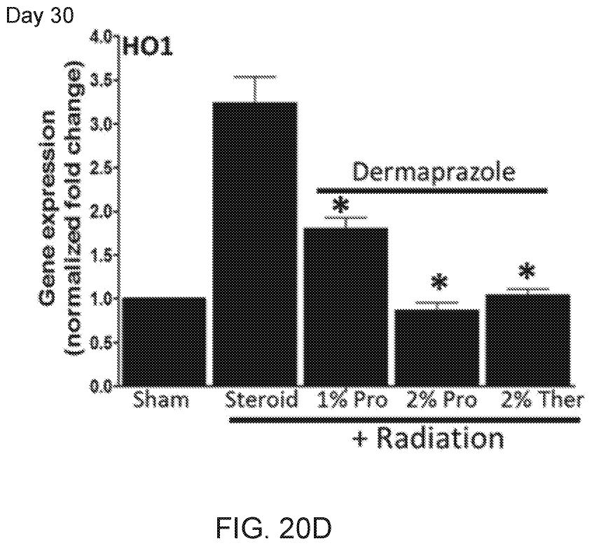

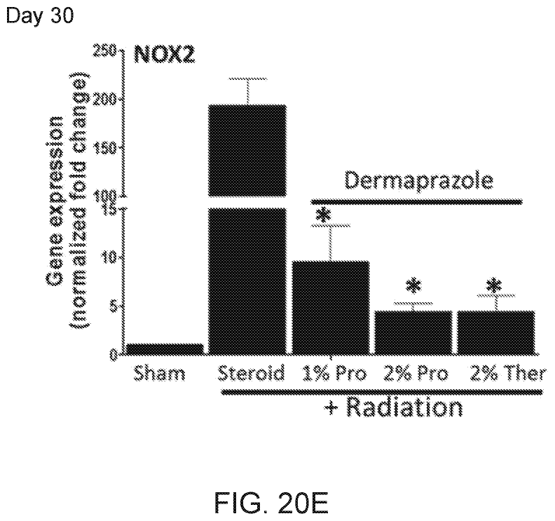

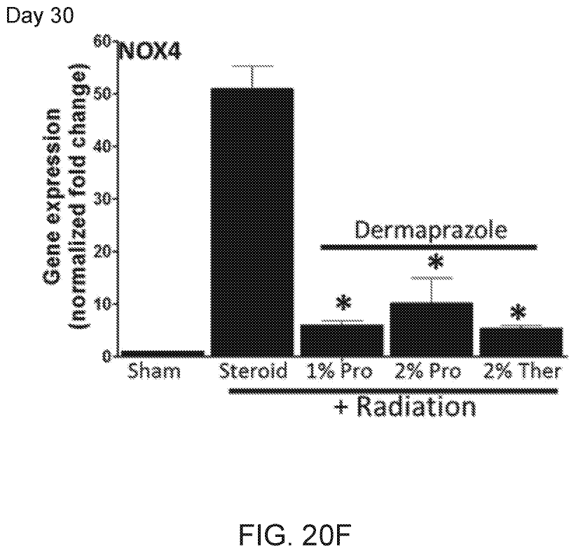

Dermaprazole temporally upregulates the expression of HO1 in dermatitis model: As expected, treatment of mice with low dose or high dose of dermaprazole in a prophylactic or therapeutic course significantly upregulated the gene expression of HO1 at the disease peak (i.e. day 16). This was mirrored by relatively low expression of the major pro-oxidant enzymes NOX2 and NOX4 (FIGS. 20A-20F). However, this upregulation of antioxidant defense mechanism was temporal and subsided after the animals have recovered from the effects of ionizing radiation (i.e. day 30). Intriguingly, however, the animals that still manifested radiation dermatitis by day 30 (e.g. steroid group) had elevated levels of HO1; a cytoprotective molecule known to be induced by cellular stress (Choi, 1996), at this time point (FIGS. 20A-20F). Consistent with the phenotype of increased cellular stress is the very high levels of NOX2 and NOX4 in the steroid treated group by day 30 (FIGS. 20A-20F). Collectively, these data sets suggest that the animals in the steroid group were still coping with the radiation-induced oxidative burden in part through heightened antioxidant defense mechanism.

### Significance of Certain Embodiments

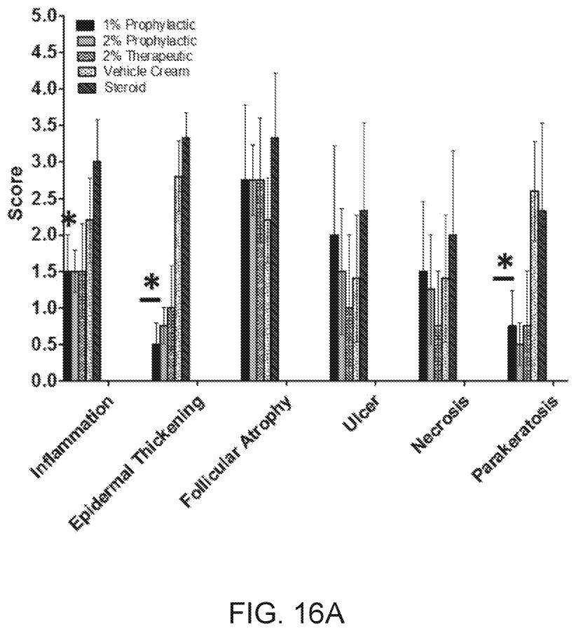

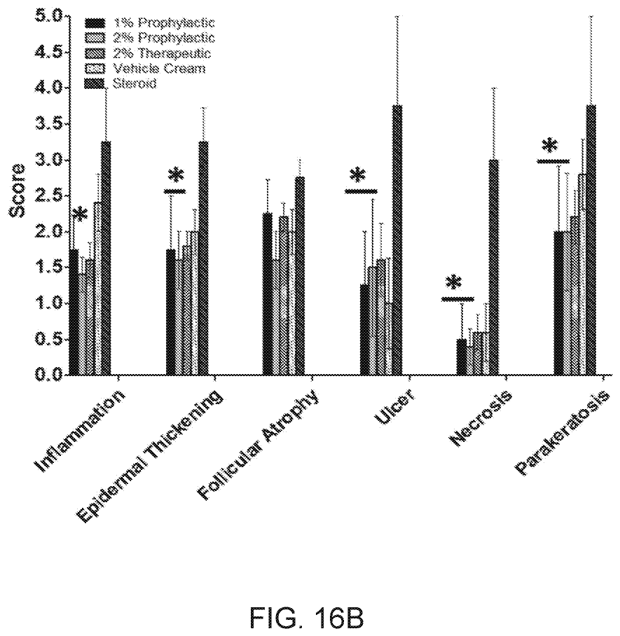

The present disclosure encompasses the repurposing of PPIs for radiation-induced or chemoradiation-induced complications. Accordingly, esomeprazole was reformulated into a topical product that is able to penetrate into the dermis and protect the skin from harmful effects of chemotherapy (bleomycin) or ionizing radiation including ulceration, necrosis, inflammation, and fibrosis (FIGS. 11, 12, 14, 16, 13, 14, 18, 25, and 26) resulting in nearly complete closure of the wounds in most of the dermaprazole-treated animals. Unexpectedly, the hair also grew back in the dermaprazole-treated animals albeit that the appearance was grayish compared to the unirradiated and untreated area of the skin (FIG. 11). Notably, it was found that the relatively low strength of dermaprazole (1-5%) was more stable and biologically more active than the higher strengths (10-20%) that were formulated. This phenomenon is likely because of exposure of the higher esomeprazole content in the formulation to air oxidation. This combined with the presence of sulphoxide moiety in the chemical structure of the compound might be an issue for the long term stability of the compound at ambient temperature.

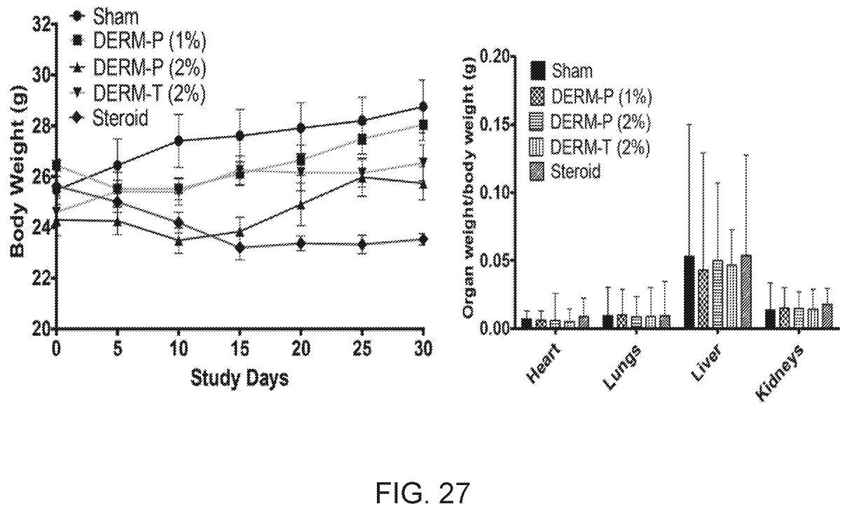

Dermaprazole retains the biological activity of esomeprazole: The formulation of esomeprazole powder into a Lipoderm®-based cream produced a topical product, dermaprazole, that retained the biological activity of esomeprazole. Esomeprazole, the S-enantiomer of omeprazole, is a PPI widely used for the treatment of gastroesophageal reflux. The antioxidant effect of PPIs is because of direct scavenging of ROS and restoration of depleted endogenous antioxidants (Biswas et al., 2003; Simon et al., 2006). The anti-inflammatory effect of PPIs is in part due to downregulation of classic pro-inflammatory molecules and impaired migration of neutrophils (Ghebremariam et al., 2015; Yoshida et al., 2000; Handa et al., 2006). More recently, it is shown that the antifibrotic effect of PPIs to be due to upregulation of HO1, downregulation of extracellular matrix components and direct inhibition of fibroblast proliferation (Ghebremariam et al., 2015). Intriguingly, all of these extra-gastric effects of PPIs could not be reproduced with other antacids such as the histamine H2-receptor antagonists (Ghebremariam et al., 2015; Yoshida et al., 2000; Ghebre et al., 2016). Accordingly, the effect of PPIs on extra-gastric targets is likely due to the presence of benzimidazole moiety in their structure. Benzimidazoles are considered as privileged scaffolds and form the basis for about 25% of the hundred most selling drugs (Khokra et al., 2011). In the present study, dermaprazole maintained the antioxidant, anti-inflammatory and antifibrotic effects that are also possessed by its unformulated analog, esomeprazole. In addition, dermaprazole was well-tolerated with no adverse effects on body weight or the weight of the heart, lungs, kidneys and liver (FIG. 27).

Dermaprazole downregulates DDAH-iNOS pathway in dermal tissue ex vivo and in vivo: Recent studies indicate that DDAH, an enzyme expressed by every nucleated mammalian cell, supports proinflammatory and profibrotic activities. For example, Pullamsetti et al (Pullamsetti et al., 2011) used a mouse model that genetically overexpresses human DDAH and showed that exposure of these transgenic mice to the chemotherapeutic drug bleomycin exaggerates the fibrotic response including greater accumulation of collagen, while DDAH inhibition with a small molecule suppresses fibrotic changes. Similarly, other studies have noted pathobiologic role of the NOS pathway associated with increased proliferation of fibroblasts and augmented fibrotic tissue remodeling (Dooley et al., 2012). Recently published data demonstrate that PPIs modulate both DDAH and iNOS (Ghebremariam et al., 2013; Ghebremariam et al., 2015), and systemic administration of esomeprazole in a mouse model of bleomycin-induced lung injury suppresses lung inflammation and fibrosis (Ghebremariam et al., 2015). Similarly, the present data shows that the expression of both DDAH and iNOS are upregulated by ionizing radiation and dermaprazole significantly downregulates their expression in the dermal tissue (FIGS. 17A-17H and FIGS. 19A-19F). Meanwhile, the profound upregulation of key biological molecules that are typically expressed by vascular endothelial cells (e.g. VCAM1, ICAM1) and immune cells (e.g. TLR4) following exposure to ionizing radiation indicates that the radiation dermatitis in this model may involve vasculopathy and immune dysfunction. Notably, dermaprazole favorably regulated expression of these molecules (FIGS. 17A-17H).

Esomeprazole and other proton pump inhibitors chemosensitize tumor cells: The use of dermaprazole in cancer patients affected with dermatitis raises the question of whether concomitant use of PPIs impair the anti-tumor effect of chemoradiation therapy in part by protecting the tumor from the anticancer treatment. Part of this concern may be addressed from existing in vitro and in vivo data available in the literature. For example, Luciani et al (2004) assessed the sensitivity of 28 chemotherapy-resistant human cancer cell lines upon pretreatment with the PPIs omeprazole and esomeprazole. They found that pretreatment of cell lines derived from melanoma, colon, breast and ovarian cancers with the PPIs resulted in order of magnitude reduction in the half maximal inhibitory concentration (IC50) values for the chemotherapeutic agents cisplatin, vinblastine and 5-fluorouracil compared to no PPI pretreatment control. In addition, their in vivo study demonstrated that pretreatment of engrafted tumor with PPI increased sensitivity of the tumor cells to cisplatin resulting in significant reduction in tumor weight. Similarly, several other studies in mice, cats and dogs have demonstrated greater sensitivity of tumor cells to anticancer drugs upon pretreatment with PPIs (Ouar et al., 2003; Spugnini et al., 2011; Patel et al., 2013; Huang et al., 2013; Ferrari et al., 2013; Lindner et al., 2014; Goh et al., 2014). However, in specific embodiments of methods of the disclosure, the PPIs are not utilized for chemosensitization of cancer cells. In alternative embodiments, the PPIs in topical form are utilized for chemosensitization of cancer cells.

Accordingly, the effect of esomeprazole on melanoma and breast cancer cell lines (as examples only) in particular indicate that treatment of dermatitis with dermaprazole in patients with these tumors may simultaneously inhibit tumor cell proliferation and increase the efficacy of anticancer therapy on the underlying tumor which may eventually lead to reducing the total chemoradiation dose prescribed to these patients on dermaprazole. Additional corroboration to this possibility comes from the observation that some melanoma cells may depend on VCAM1, a molecule significantly downregulated by dermaprazole (FIGS. 17A-17H), to adhere to vasculature (Eibl et al., 2004).

Proton pump inhibitors prolong survival in cancer patients: In line with the increased sensitivity of solid tumor-derived cancer cells to chemotherapeutic drugs upon PPI treatment in preclinical models, clinical studies also reported that PPIs are associated with beneficial outcomes including prolonged survival. In a retrospective cohort of 596 patients with head and neck squamous cell carcinomas (HNSCC), Papagerakis et al (2014) found that patients taking PPIs had significantly longer overall survival compared to patients on convention treatment. Similarly, other studies also found that supplementation of standard cancer care with PPIs is associated with increased progression-free survival and overall survival (Wang et al., 2017). Currently, there are completed or ongoing clinical trials evaluating PPIs as adjuvants in cancer (e.g. NCT01069081). Given that one of the major challenges of HNSCC treatment is resistance to standard of care and poor overall survival, readily available and well tolerated drugs such as PPIs in certain embodiments are useful. In addition, topical PPI such as dermaprazole has clinical utility in HNSCC to reduce common adverse effects of chemoradiation therapy including mucositis, and potentially improve the quality and quantity of life, in specific embodiments

In conclusion, dermaprazole, the topically formulated PPI esomeprazole, is able to macroscopically and microscopically reduce inflammation and scarring induced by chemotherapy or ionizing radiation in preclinical models of dermal inflammation and fibrosis. The anti-inflammatory and antifibrotic effects of dermaprazole in the radiation dermatitis model in specific embodiments are because of the early induction of endogenous antioxidant defense mechanisms and downregulation of pro-inflammatory and profibrotic mechanisms. Some of the dermaprazole-regulated key molecular pathways include HO1-Nrf2, DDAH-iNOS and collagen. Early induction of the HO1 and Nrf2 molecules by dermaprazole is expected to prepare the skin tissue to handle the early surge in oxidative stress perpetrated by chemotherapy or ionizing radiation. Subsequently, downstream targets of ROS, superoxide and hydroxyl radicals including pro-inflammatory and profibrotic cytokines are tightly regulated. The remarkable efficacy of dermaprazole on dermal tissue inflammation and fibrosis, as well as the antitumor activity of PPIs render dermaprazole useful to reduce cancer therapy-induced inflammation while chemo/radio sensitizing the underlying tumor, in certain embodiments. In other embodiments, dermaprazole is useful in other skin conditions characterized by inflammatory and/or fibrotic phases such as scleroderma, atopic dermatitis, seborrheic dermatitis, and rheumatic diseases. In particular, the profound effect of dermaprazole on classic proinflammatory molecules such as TNFα, innate immune signaling such as TLR4, and components of extracellular matrix (ECM) such as collagen and fibronectin (FIGS. 17A-17H, 18, 19A-19F) indicates efficacy of the formulation beyond radiation-induced ailments into diseases of the connective tissue, in particular embodiments.

Examples of Materials and Methods

Formulation of esomeprazole: To develop esomeprazole into a topical product for potential application in radiation-induced inflammatory and fibrotic skin conditions, the inventors compounded esomeprazole powder into a cream. In brief, esomeprazole powder (>98.5% purity) was weighed and placed in a mortar. To enable epidermal penetration, the powder was wetted with propylene glycol and mixed with Lipoderm (60%)/vanishing cream (40%) transdermal base. Finally, the product (i.e. dermaprazole) was run through an ointment mill to minimize grittiness and dispensed into small containers. Using this protocol, the inventors made different formulations including vehicle cream (cream base only) and dermaprazole at 0.01%, 0.1%, 1%, 2%, 3%, 4%, 5%, 10%, and 20%. The odor, appearance and color of the formulations were assessed on days 1, 15 and 30. Odor was measured as described in the European Pharmacopoeia, i.e. by spreading 1.5 gram of cream on a 60 mm watch glass and smelling the cream after 15 minutes. Appearance and color was referenced to day 1 for any changes. Drug content was quantified using liquid chromatography-mass spectrometry (LC-MS) as described below.

Characterizing physicochemical stability of dermaprazole: LC-MS and Atomic Force Microscopy (AFM) was used to characterize dermaprazole. For the LC-MS study, esomeprazole content was measured in the cream in the presence of cream-only control. In brief, 200 μl of water/methanol (v/v 1:1) was added to 10 mg of cream. For 10% and 20% dermaprazole, 250 μl of water/methanol (v/v 1:4) was added to 10 mg of the cream. The resulting mixture was vortexed for 5 minutes. Ice-cold methanol (150 μl) with internal standard (IS) was added to 50 μl of the resulting mixture from placebo, 0.01% and 0.1% cream and to 25 μl of the mixture from 1% cream, followed by centrifugation at 15000 g for 15 minutes. The mixture from 10% and 20% cream obtained from above were further diluted 16 and 32 times with methanol, respectively. Ice-cold methanol (150 μl) with IS was added to 50 μl of the diluted mixture from 10% and 20% cream, followed by centrifugation at 15000 g for 15 minutes. The supernatant from placebo, 0.01%, 0.1% cream were directly transferred to the sample vial for analysis. The supernatant from the 1%, 10% and 20% cream were further diluted 100× in methanol with IS. After 5 minutes of centrifugation, the supernatant was transferred to sample vials for analysis.

The prepared samples (5 μl each) were injected into an HPLC-MS/MS (Agilent Technologies, 6490 QQQ Santa Clara, Calif.) for analysis. Esomeprazole separation was achieved using a 1290 Infinity LC System (Agilent Technologies, Santa Clara, Calif.) equipped with a 50×2.1 mm (Agilent ZORBAX SB-Aq) column. The column temperature was maintained at 40° C. The flow rate was 0.3 mL/min with a gradient in an 8 minute run. Gradients were run starting from 90% buffer A (H2O with 0.1% formic acid) to 50% buffer B (CH3CN with 0.1% formic acid) from 0 to 6 minutes; 50% buffer B was held for 0.5 minutes; 50% buffer B to 90% buffer A from 6.5 to 7 minute and 90% buffer A held from 7 to 8 minute to re-equilibrate the column. LC-MS/MS was operated in positive mode with electrospray ionization (ESI) with multiple reaction monitoring (MRM) mode. Ultra-highly pure nitrogen was applied as the drying gas and the collision gas. The precursor to production transitions for dermaprazole and esomeprazole IS control were as follows: m/z 346.1>198.1 for EL20; m/z 244.1>185.3 for IS. Mass chromatograms and mass spectra were acquired by MassHunter Workstation data Acquisition software (Agilent, Santa Clara, Calif.) and the data were analyzed by triple quadrupole (QQQ)

Quantitative Analysis software (Agilent, Santa Clara, Calif.). The extract ion chromatogram peak integration were performed by the agile 2 integrator that is included in the Quantitative Analysis software. Meanwhile, the topography, stiffness and adhesion of dermaprazole were studied by spreading the cream onto microscopic slides and examination using an ultra-high resolution material science microscope, MultiMode Atomic Force Microscope (Bruker Corporation, Billerica, Mass.).

Drug permeation study: The ex vivo release of esomeprazole from dermaprazole formulation was studied using mice skin excised from the abdominal area. In brief, C57 mice were euthanized by CO2 inhalation and bilateral opening of the thorax prior to removing hair from the abdomen area. Subsequently, a piece of the abdominal skin was excised and the underlying fascia was separated using a surgical scissor. Maintenance of the integrity of the skin and removal of any residual fat and subcutaneous tissue were visually confirmed. The prepared skin was wrapped in aluminum foil and stored at −80° C. until use. Ex vivo skin permeation studies were conducted using Franz Diffusion Cell (PermeGear, Hellertown, Pa.) (Ng et al., 2010). Briefly, the excised skin was mounted between the donor and receptor compartments of the cell exposing an area of 0.64 cm² with sink volume of 5 mL. The receptor compartment contained phosphate buffer saline (PBS, pH 7.4) as diffusion medium to maintain sink condition and its temperature was maintained at 37±0.5° C. The prepared formulations (40 mg each) were applied to the skin in the donor compartment and uniformly spread using cotton-tipped applicator. Hourly, 20 μL of sample was removed from the sampling port of the Diffusion Cell and equal amount of PBS was added to maintain constant total volume. The release of esomeprazole into the receptor chamber was measured over time using a spectrophotometer.

Ex vivo study: 3D human skin model: For this study, three-dimensional (3D) human skin model (EpiDermFT, MatTek Corporation, Ashland, Mass.) was cultured in tissue culture system following the supplier's recommendation. In brief, the EpiDermFT was supplied as single well tissue culture plate inserts with each insert containing functionally and metabolically active reconstituted skin with surface area of 1.0 cm² (von Neubeck et al., 2012). Upon receipt, the EpiDermFT was equilibrated in EFT media at 37° C., 5% CO2 for 24 hours. Throughout the experiment, EpiDermFT was maintained in 6 well culture plates at air liquid interface with dermal side of the tissue contacting the tissue culture media and the epidermal stratum corneum side exposed to air. After 24 hours of incubation, the culture was replenished with fresh media and the skin was subsequently exposed to 14 Gray (Gy) of ionizing (X-ray) radiation at 30 cm source-to-skin distance and at a rate of 1.2 Gy/min (160 kV) using RS-2000 Biological System. Next, the tissue was incubated in the same condition for another hour prior to applying vehicle cream (negative control), dermaprazole (1% to 20%) or 1% hydrocortisone (positive control) to the apical surface. After 24 hours of topical treatment, the tissues were homogenized and total RNA was isolated using the Direct-zol RNA Miniprep Kit (Zymo Research, Irvine, Calif.). Subsequently, the concentration and quality of RNA was verified and 1 μg of total RNA was reverse transcribed using High Capacity RNA-to-cDNA kit (Applied Biosystems, Foster City, Calif.). The resulting cDNA was used for gene expression study by quantitative RT-PCR. Quantitative RT-PCR (qRT-PCR) was performed using standard TaqMan gene expression assay using inventoried “best coverage” primer/probe sets (ThermoFisher Scientific, Waltham, Mass.) as described below.

In vivo study: mouse model of radiation-induced dermatitis: a 30-day study was conducted using C57BL/6J mice (Jackson Laboratories, Bar Harbor, Me.) to assess the efficacy of dermaprazole in mitigating radiation-induced dermatitis. The experiment consisted of 1 group receiving no radiation (Group 1) and 5 groups receiving radiation (Groups 2 to 6) (Table 2) from an X-ray source.

TABLE 2
Experimental design of ionizing radiation (IR)-induced dermatitis in a 30-day mouse model.
The groups were exposed to control or IR in the flank and received vehicle, low (1%) or
high (2%) dermaprazole (DERM) cream in a prophylactic or therapeutic course.
The corticosteroid hydrocortisone (1%) was included as a treatment control.

| | | | Radiation | | | | | |
|---|---|---|---|---|---|---|---|---|
| Group | | | Dose (Gy), | Treatment | | Dosing | Dermatitis | Necropsy |
| ID | Exposure | N | Route | Cream | Route | Schedule | Evaluation | Day |
| 1 | Sham Control | 5 | 0, Flank | Vehicle | Topical | 0-30 | Day 4-30 | 16, 30 |
| 2 | IR + DERM | 10 | 2 × 15Gy, | 1% DERM | Topical | 0-30 | Day 4-30 | 16, 30 |
| | 1% Prophylactic | | Flank | | | | | |
| 3 | IR + DERM | 10 | 2 × 15Gy, | 2% DERM | Topical | 0-30 | Day 4-30 | 16, 30 |
| | 2% Prophylactic | | Flank | | | | | |
| 4 | IR + DERM | 10 | 2 × 15Gy, | 2% DERM | Topical | 10-30 | Day 4-30 | 16, 30 |
| | 2% Therapeutic | | Flank | | | | | |
| 5 | IR Control | 10 | 2 × 15Gy, | Vehicle | Topical | 0-30 | Day 4-30 | 16, 30 |
| | | | Flank | | | | | |
| 6 | IR + | 10 | 2 × 15Gy, | 1% | Topical | 0-30 | Day 4-30 | 16, 30 |
| | Corticosteroid | | Flank | hydrocortisone | | | | |

N = number of animals.
Gy= radiation dose in Gray

First, all the animals were prepared for experiment by removing hair from the flank area using an electric shaver and hair removal cream. The lack of skin irritation from the shaving procedure was visually confirmed prior to randomization. Twenty-four hours later, radiation was delivered to pre-defined area (2×2 cm) on the flank of the animals in Groups 2 to 6. To induce dermatitis, the animals were anesthetized using 4% isoflurane in oxygen until a deep plane of anesthesia was achieved. Subsequently, the animals were placed under lead shielding (Braintree Scientific, Braintree, Mass.) to protect the remaining part of the body and expose the flank to a total radiation dose of 30 Gy fractionated as 2×15 Gy on study days 0 and 7 using the same settings described above. Dermaprazole was evaluated in a prophylactic (treatment schedule from day 1 to day 30) and therapeutic (treatment schedule from day 10 to day 30) course. For the prophylactic course, low dose (1%, Group 2) and high dose (2%, Group 3) of dermaprazole were evaluated. For the therapeutic course (Group 4), only the high dose was evaluated. Similarly, the sham (Group 1) and vehicle (Group 5) groups were treated with placebo cream containing no active drug starting from day 0 of the study. As a positive treatment control, 1% hydrocortisone (Group 6) was applied starting from day 0. On the days of irradiation, the topical treatments were applied one hour later. All the animals were treated once a day until euthanasia.

To monitor dermatitis severity, digital photographs were captured (Canon PowerShot ELPH 180) under the same settings at baseline, day 16 (the anticipated time to peak of injury), and day 30 (completion of the study). The photographs were blindly evaluated and scored by a certified dermatologist using Common Terminology Criteria for Adverse Events (CTCAE; version 4.0)(79): 0=normal skin appearance; 1=mild erythema; 2=moderate-to-severe erythema; 3=desquamation of 25-50% of irradiated area; 4=desquamation of >50% irradiated area; and 5=frank ulcer. On day 16, about 3 hours after topical treatment, 5 animals per group (except the sham) were euthanized and skin samples together with underlying muscle tissue were harvested from the irradiated area for gene expression and histopathological studies. Similarly, all the remaining animals were euthanized on day 30 of the study. For consistency, the irradiated skin of the animals was divided into two portions by vertical dissection where the right side was used for histopathological studies, and the left side was used for gene and protein expression studies. For the later studies, RNA and protein were isolated from the same samples using the NucleoSpin RNA/Protein kit (Macherey-Nagel, Bethlehem, Pa.) following the manufacturer's recommendation

Gene expression study: For gene expression study, cDNA was generated as described above and was used for qRT-PCR to compare the effect of dermaprazole on the mRNA expression of genes that are reported to play significant role in oxidative stress, inflammation and/or fibrosis including: Nrf2, HO1, NADPH oxidase 2 (NOX2), NOX4, iNOS, DDAH 1, TNFα, NFκB, TGFβ, toll-like receptor 4 (TLR4), interleukin 1 beta (IL1β), interleukin 6 (IL6), intercellular adhesion molecule 1 (ICAM1), vascular cell adhesion molecule 1 (VCAM1), collagen (1, 3, 5), and fibronectin. Ribosomal RNA 18S (18S rRNA) was used as internal control for this assay. Quantitative RT-PCR was performed in a 96-well plate in 20 μL final volume containing 10 μL of TaqMan Universal PCR Master mix (2×), 1 μL of TaqMan assay containing primers and MGB probe mix (20×), 3 μL of cDNA and 6 μL of water. The reaction was carried out under the following condition: incubation at 50° C. for 2 minutes; denaturation at 95° C. for 10 minutes followed by 95° C. for 15 seconds and annealing and extension at 60° C. for 1 minute for 40 cycles in total. The run was performed using the CFX Real-Time PCR System (Bio-Rad) and the data was analyzed using CFX Maestro Software. Data is shown as relative gene expression to sham control after normalizing to 18S.

Protein expression study: Cytoplasmic and nuclear proteins were isolated from the 3D human skin using NE-PER Nuclear and Cytoplasmic Extraction Reagents Kit (ThermoFisher Scientific). Protein concentration was quantified using Pierce™ BCA Protein Assay Kit (ThermoFisher Scientific) and 30 μg of each protein was loaded for Western blot analyses. The effect of dermaprazole on the expression of the nuclear protein Nrf2 (rabbit anti-Nrf2; Abcam ab62352; 1:250) and the cytoplasmic protein HO1 (rabbit anti-HO1; Enzo Life Sciences BML-HC3001; 1:500) was assessed in comparison to vehicle and corticosteroid controls. Histone H3 (rabbit anti-H3; Abcam ab1791; 1:3000) was used as internal control for the nuclear protein expression and GAPDH (mouse anti-GAPDH; ThermoFisher MA5-157381:5000) was used as a control for the cytoplasmic protein expression. For the animal tissue samples, total protein was isolated using the NucleoSpin RNA/Protein kit as described above.

Histopathological study: For this study, biopsies from the right side of the irradiated skin were fixed in 10% neutral buffered formalin and processed to slides for immunohistochemistry. Fixed tissues were paraffin embedded and sectioned at 4 μm thickness. One set of slides was stained with H&E for the assessment of inflammation and overall tissue architecture; another set was stained with Masson's Trichrome to examine the degree of collagen deposition and fibrotic changes. Two additional slide sets were used to stain for the inflammatory cell markers CD11b (myeloid leukocytes; Abcam ab133357; 1:1000) and F4/80 (macrophages; Cell Signaling 70076S; 1:250). H&E and Trichrome stained slides were examined microscopically and graded for the degree of inflammation and fibrosis respectively. A board-certified dermatopathologist scored the tissues in a blinded fashion based on a 5-point scale for epidermal thickening, follicular atrophy/hypertrophy, inflammation, collagen thickening, ulcer, necrosis and parakeratosis/crust. For each of the parameters, scores were defined as: 0=normal skin; 1=minimally detectable; 2=mild; 3=moderate; 4=marked and 5=severe.

Mouse model of scleroderma: To complement the above studies, the efficacy of dermaprazole was evaluated in a mouse model of scleroderma; a disease characterized by collagen accumulation. For this, the established mouse model of bleomycin-induced dermal fibrosis (54, 55) was used where B.10.A. mice (Jackson Laboratories) (n=4/group) were subjected to subcutaneous injection of bleomycin sulfate (3.3 mg/kg/day) for the first 4 weeks of the study. Starting from day 21 of the study, the animals were treated topically (1×/day) with aquaphor, corticosteroid (0.1% mometasone furoate), or dermaprazole (1% or 2%) until necropsy. Photographic images of the wound site were taken for comparison. In addition, skin tissues were mounted on microscopic slides and stained for Hematoxylin and Eosin (H&E), and Masson's Trichrome.

Statistical Analysis

The number of animals per study group was calculate using power and sample size calculation (PS; Vanderbilt University). Both parametric and nonparametric data was analyzed by one-way ANOVA (GraphPad prism; La Jolla, Calif.) unless indicated otherwise. Multiple groups were compared using ANOVA followed by Bonferroni posthoc test and differences between two groups were compared using unpaired t test. All data are expressed as Mean±SEM unless indicated otherwise. Differences are considered statistically significant at a value of p below 0.05 (p<0.05).

### REFERENCES

Biswas, K., U. Bandyopadhyay, I. Chattopadhyay, A. Varadaraj, E. Ali, R. K. Banerjee, A novel antioxidant and antiapoptotic role of omeprazole to block gastric ulcer through scavenging of hydroxyl radical. J Biol Chem. 278, 10993-1001 (2003).
Bostrom, A., H. Lindman, C. Swartling, B. Berne, J. Bergh, Potent corticosteroid cream (mometasone furoate) significantly reduces acute radiation dermatitis: results from a double-blind, randomized study. Radiother Oncol. 59, 257-65 (2001).
Brach, M. A., H. J. Gruss, T. Kaisho, Y. Asano, T. Hirano, F. Herrmann, Ionizing radiation induces expression of interleukin 6 by human fibroblasts involving activation of nuclear factor-kappa B. J Biol Chem. 268, 8466-72 (1993).
Brown K. R., E. Rzucidlo, Acute and chronic radiation injury. J Vasc Surg. 53, 15S-21S (2011).
Campbell, I. R., M. H Illingworth, Can patients wash during radiotherapy to the breast or chest wall? A randomized controlled trial. Clin Oncol (R Coll Radiol). 4, 78-82 (1992).
Chan, R. J., J. Webster, B. Chung, L. Marquart, M. Ahmed, S. Garantziotis, Prevention and treatment of acute radiation-induced skin reactions: a systematic review and meta-analysis of randomized controlled trials. BMC Cancer. 14, 53 (2014).
Chen, A. P., A. Setser, M. J. Anadkat, J. Cotliar, E. A. Olsen, B. C. Garden, M. E. Lacouture, Grading dermatologic adverse events of cancer treatments: the Common Terminology Criteria for Adverse Events Version 4.0. J Am Acad Dermatol. 67, 1025-39 (2012).
Chen, Y. P., N. M. Tsang, C. K. Tseng, S. Y. Lin, Causes of interruption of radiotherapy in nasopharyngeal carcinoma patients in Taiwan. Jpn J Clin Oncol. 30, 230-4 (2000).
Coondoo, A., M. Phiske, S. Verma, K. Lahiri, Side-effects of topical steroids: A long overdue revisit. Indian Dermatol Online J. 5, 416-25 (2014).
Cox, J. D., J. Stetz, T. F. Pajak, Toxicity criteria of the Radiation Therapy Oncology Group (RTOG) and the European Organization for Research and Treatment of Cancer (EORTC). Int J Radiat Oncol Biol Phys. 31, 1341-6 (1995).
Dooley, A., K. R. Bruckdorfer, D. J. Abraham, Modulation of fibrosis in systemic sclerosis by nitric oxide and antioxidants. Cardiol Res Pract. 2012, 521958 (2012).
Eibl, R. H., M. Benoit, Molecular resolution of cell adhesion forces. IEE Proc Nanobiotechnol. 151, 128-32 (2004).
Elliott, E. A., J. R. Wright, R. S. Swann, F. Nguyen-Tan, C. Takita, M. K. Bucci, A. S. Garden, H. Kim, E. B. Hug, J. Ryu, M. Greenberg, J. P. Saxton, K. Ang, L. Berk, T. Radiation Therapy Oncology Group, Phase III Trial of an emulsion containing trolamine for the prevention of radiation dermatitis in patients with advanced squamous cell carcinoma of the head and neck: results of Radiation Therapy Oncology Group Trial 99-13. J Clin Oncol. 24, 2092-7 (2006).
Ferrari, S., F. Perut, F. Fagioli, A. Brach Del Prever, C. Meazza, A. Parafioriti, P. Picci, M. Gambarotti, S. Avnet, N. Baldini, S. Fais, Proton pump inhibitor chemosensitization in human osteosarcoma: from the bench to the patients' bed. J Transl Med. 11, 268 (2013).
Gamulin, M., V. Garaj-Vrhovac, N. Kopjar, Evaluation of DNA damage in radiotherapy-treated cancer patients using the alkaline comet assay. Coll Antropol. 31, 837-45 (2007).
Ghebre Y. T., G. Raghu, Idiopathic Pulmonary Fibrosis: Novel Concepts of Proton Pump Inhibitors as Antifibrotic Drugs. Am J Respir Crit Care Med. 193, 1345-52 (2016).
Ghebremariam, Y. T., J. P. Cooke, W. Gerhart, C. Griego, J. B. Brower, M. Doyle-Eisele, B. C. Moeller, Q. Zhou, L. Ho, J. de Andrade, G. Raghu, L. Peterson, A. Rivera, G. D. Rosen, Pleiotropic effect of the proton pump inhibitor esomeprazole leading to suppression of lung inflammation and fibrosis. J Transl Med. 13, 249 (2015).
Ghebremariam, Y. T., P. LePendu, J. C. Lee, D. A. Erlanson, A. Slaviero, N. H. Shah, J. Leiper, J. P. Cooke, Unexpected effect of proton pump inhibitors: elevation of the cardiovascular risk factor asymmetric dimethylarginine. Circulation. 128, 845-53 (2013).
Glees, J. P., H. Mameghan-Zadeh, C. G. Sparkes, Effectiveness of topical steroids in the control of radiation dermatitis: a randomised trial using 1% hydrocortisone cream and 0.05% clobetasone butyrate (Eumovate). Clin Radiol. 30, 397-403 (1979).
Goh, W., I. Sleptsova-Freidrich, N. Petrovic, Use of proton pump inhibitors as adjunct treatment for triple-negative breast cancers. An introductory study. J Pharm Sci. 17, 439-46 (2014).
Hainan, K. E., The effect of corticosteroids on the radiation skin reaction. A random trial to assess the value of local application of prednisolone and neomycin ointment after x-ray treatment of basal cell carcinoma. Br J Radiol. 35, 403-8 (1962).
Handa, O., N. Yoshida, N. Fujita, Y. Tanaka, M. Ueda, T. Takagi, S. Kokura, Y. Naito, T. Okanoue, T. Yoshikawa, Molecular mechanisms involved in anti-inflammatory effects of proton pump inhibitors. Inflamm Res. 55, 476-80 (2006).
Ho, A. Y., M. Olm-Shipman, Z. Zhang, C. T. Siu, M. Wilgucki, A. Phung, B. B. Arnold, M. Porinchak, M. Lacouture, B. McCormick, S. N. Powell, D. Y. Gelblum, A Randomized Trial of Mometasone Furoate 0.1% to Reduce High-Grade Acute Radiation Dermatitis in Breast Cancer Patients Receiving Postmastectomy Radiation. Int J Radiat Oncol Biol Phys. 101, 325-333 (2018).
Huang, S., M. Chen, X. Ding, X. Zhang, X. Zou, Proton pump inhibitor selectively suppresses proliferation and restores the chemosensitivity of gastric cancer cells by inhibiting STATS signaling pathway. Int Immunopharmacol. 17, 585-92 (2013).
Khokra S L, Choudhary D, Benzimidazole An Important Scaffold In Drug Discovery. Asian Journal of Biochemical and Pharmaceutical Research. 1, 476-486 (2011).
Lindner, K., C. Borchardt, M. Schopp, A. Burgers, C. Stock, D. J. Hussey, J. Haier, R. Hummel, Proton pump inhibitors (PPIs) impact on tumour cell survival, metastatic potential and chemotherapy resistance, and affect expression of resistance-relevant miRNAs in esophageal cancer. J Exp Clin Cancer Res. 33, 73 (2014).
Lomax, M. E., L. K. Folkes, P. O'Neill, Biological consequences of radiation-induced DNA damage: relevance to radiotherapy. Clin Oncol (R Coll Radiol). 25, 578-85 (2013).
Lopez, E., R. Guerrero, M. I. Nunez, R. del Moral, M. Villalobos, J. Martinez-Galan, M. T. Valenzuela, J. A. Munoz-Gamez, F. J. Oliver, D. Martin-Oliva, J. M. Ruiz de Almodovar, Early and late skin reactions to radiotherapy for breast cancer and their correlation with radiation-induced DNA damage in lymphocytes. Breast Cancer Res. 7, R690-8 (2005).
Luciani, F., M. Spada, A. De Milito, A. Molinari, L. Rivoltini, A. Montinaro, M. Marra, L. Lugini, M. Logozzi, F. Lozupone, C. Federici, E. Iessi, G. Parmiani, G. Arancia, F. Belardelli, S. Fais, Effect of proton pump inhibitor pretreatment on resistance of solid tumors to cytotoxic drugs. J Natl Cancer Inst. 96, 1702-13 (2004).
Macmillan, M. S., M. Wells, S. MacBride, G. M. Raab, A. Munro, H. MacDougall, Randomized comparison of dry dressings versus hydrogel in management of radiation-induced moist desquamation. Int J Radiat Oncol Biol Phys. 68, 864-72 (2007).
Mendelsohn, F. A., C. M. Divino, E. D. Reis, M. D. Kerstein, Wound care after radiation therapy. Adv Skin Wound Care. 15, 216-24 (2002).
McCloskey, S. A., W. Jaggernauth, N. R. Rigual, W. L. Hicks, Jr., S. R. Popat, M. Sullivan, T. L. Mashtare, Jr., M. K. Khan, T. R. Loree, A. K. Singh, Radiation treatment interruptions greater than one week and low hemoglobin levels (12 g/dL) are predictors of local regional failure after definitive concurrent chemotherapy and intensity-modulated radiation therapy for squamous cell carcinoma of the head and neck. Am J Clin Oncol. 32, 587-91 (2009).
Miller, R. C., D. J. Schwartz, J. A. Sloan, P. C. Griffin, R. L. Deming, J. C. Anders, T. J. Stoffel, R. E. Haselow, P. L. Schaefer, J. D. Bearden, 3rd, P. J. Atherton, C. L. Loprinzi, J. A. Martenson, Mometasone furoate effect on acute skin toxicity in breast cancer patients receiving radiotherapy: a phase III double-blind, randomized trial from the North Central Cancer Treatment Group N06C4. Int J Radiat Oncol Biol Phys. 79, 1460-6 (2011).
Mukherjee, D., P. J. Coates, S. A. Lorimore, E. G. Wright, Responses to ionizing radiation mediated by inflammatory mechanisms. J Pathol. 232, 289-99 (2014).
Muller K., V. Meineke, Radiation-induced alterations in cytokine production by skin cells. Exp Hematol. 35, 96-104 (2007).
Ng, S. F., J. J. Rouse, F. D. Sanderson, V. Meidan, G. M. Eccleston, Validation of a static Franz diffusion cell system for in vitro permeation studies. AAPS PharmSciTech. 11, 1432-41 (2010).
Okunieff, P., J. Xu, D. Hu, W. Liu, L. Zhang, G. Morrow, A. Pentland, J. L. Ryan, I. Ding, Curcumin protects against radiation-induced acute and chronic cutaneous toxicity in mice and decreases mRNA expression of inflammatory and fibrogenic cytokines. Int J Radiat Oncol Biol Phys. 65, 890-8 (2006).
Omidvari, S., H. Saboori, M. Mohammadianpanah, A. Mosalaei, N. Ahmadloo, M. A. Mosleh-Shirazi, F. Jowkar, S. Namaz, Topical betamethasone for prevention of radiation dermatitis. Indian J Dermatol Venereol Leprol. 73, 209 (2007).
Ouar, Z., M. Bens, C. Vignes, M. Paulais, C. Pringel, J. Fleury, F. Cluzeaud, R. Lacave, A. Vandewalle, Inhibitors of vacuolar H+-ATPase impair the preferential accumulation of daunomycin in lysosomes and reverse the resistance to anthracyclines in drug-resistant renal epithelial cells. Biochem J. 370, 185-93 (2003).
Papagerakis, S., E. Bellile, L. A. Peterson, M. Pliakas, K. Balaskas, S. Selman, D. Hanauer, J. M. Taylor, S. Duffy, G. Wolf, Proton pump inhibitors and histamine 2 blockers are associated with improved overall survival in patients with head and neck squamous carcinoma. Cancer Prev Res (Phila). 7, 1258-69 (2014).
Patel, K. J., C. Lee, Q. Tan, I. F. Tannock, Use of the proton pump inhibitor pantoprazole to modify the distribution and activity of doxorubicin: a potential strategy to improve the therapy of solid tumors. Clin Cancer Res. 19, 6766-76 (2013).
Pullamsetti, S. S., R. Savai, R. Dumitrascu, B. K. Dahal, J. Wilhelm, M. Konigshoff, D. Zakrzewicz, H. A. Ghofrani, N. Weissmann, O. Eickelberg, A. Guenther, J. Leiper, W. Seeger, F. Grimminger, R. T. Schermuly, The role of dimethylarginine dimethylaminohydrolase in idiopathic pulmonary fibrosis. Sci Transl Med. 3, 87ra53 (2011).
Putora, P. M., M. Schmuecking, D. Aebersold, L. Plasswilm, Compensability index for compensation radiotherapy after treatment interruptions. Radiat Oncol. 7, 208 (2012).
Richardson, J., J. E. Smith, M. McIntyre, R. Thomas, K. Pilkington, Aloe vera for preventing radiation-induced skin reactions: a systematic literature review. Clin Oncol (R Coll Radiol). 17, 478-84 (2005).
Roy, I., A. Fortin, M. Larochelle, The impact of skin washing with water and soap during breast irradiation: a randomized study. Radiother Oncol. 58, 333-9 (2001).
Schmuth, M., M. A. Wimmer, S. Hofer, A. Sztankay, G. Weinlich, D. M. Linder, P. M. Elias, P. O. Fritsch, E. Fritsch, Topical corticosteroid therapy for acute radiation dermatitis: a prospective, randomized, double-blind study. Br J Dermatol. 146, 983-91 (2002).
Simon, W. A., E. Sturm, H. J. Hartmann, U. Weser, Hydroxyl radical scavenging reactivity of proton pump inhibitors. Biochem Pharmacol. 71, 1337-41 (2006).
Sitton, E., Early and late radiation-induced skin alterations. Part II: Nursing care of irradiated skin. Oncol Nurs Forum. 19, 907-12 (1992).
Spugnini, E. P., A. Baldi, S. Buglioni, F. Carocci, G. M. de Bazzichini, G. Betti, I. Pantaleo, F. Menicagli, G. Citro, S. Fais, Lansoprazole as a rescue agent in chemoresistant tumors: a phase I/II study in companion animals with spontaneously occurring tumors. J Transl Med. 9, 221 (2011).
Trotti, A., R. Byhardt, J. Stetz, C. Gwede, B. Corn, K. Fu, L. Gunderson, B. McCormick, M. Morrisintegral, T. Rich, W. Shipley, W. Curran, Common toxicity criteria: version 2.0. an improved reference for grading the acute effects of cancer treatment: impact on radiotherapy. Int J Radiat Oncol Biol Phys. 47, 13-47 (2000).
Ulff, E., M. Maroti, J. Serup, U. Falkmer, A potent steroid cream is superior to emollients in reducing acute radiation dermatitis in breast cancer patients treated with adjuvant radiotherapy. A randomised study of betamethasone versus two moisturizing creams. Radiother Oncol. 108, 287-92 (2013).
U.S. DEPARTMENT OF HEALTH AND HUMAN SERVICES. Common Terminology Criteria for Adverse Events (CTCAE) Version 4.0. National Cancer Institute, (2009).
von Neubeck, C., H. Shankaran, N. J. Karin, P. M. Kauer, W. B. Chrisler, X. Wang, R. J. Robinson, K. M. Waters, S. C. Tilton, M. B. Sowa, Cell type-dependent gene transcription profile in a three-dimensional human skin tissue model exposed to low doses of ionizing radiation: implications for medical exposures. Environ Mol Mutagen. 53, 247-59 (2012).
Wells, M., M. Macmillan, G. Raab, S. MacBride, N. Bell, K. MacKinnon, H. MacDougall, L. Samuel, A. Munro, Does aqueous or sucralfate cream affect the severity of erythematous radiation skin reactions? A randomised controlled trial. Radiother Oncol. 73, 153-62 (2004).
Yoshida, N., T. Yoshikawa, Y. Tanaka, N. Fujita, K. Kassai, Y. Naito, M. Kondo, A new mechanism for anti-inflammatory actions of proton pump inhibitors—inhibitory effects on neutrophil-endothelial cell interactions. Aliment Pharmacol Ther. 14 Suppl 1, 74-81 (2000).
Wang, X., C. Liu, J. Wang, Y. Fan, Z. Wang, Y. Wang, Proton pump inhibitors increase the chemosensitivity of patients with advanced colorectal cancer. Oncotarget. 8, 58801-58808 (2017).

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of treating or preventing cancer therapy-induced tissue inflammation, dermatitis, scarring, dermal fibrosis, graft vs. host disease of the skin, scleroderma, Mixed Connective Tissue Disease (MCTD), Rheumatoid Arthritis (RA), lupus, polymyositis, dermatomyositis, Sjogren's Syndrome, Raynaud Disease, oral mucositis; non-oral mucositis; proctitis, enteritis, colitis, esophagitis, urinary tract inflammation, burns of any kind, frostbite or cold injury, chemical-induced dermatitis, skin graft, transplanted tissue, plastic or cosmetic surgery, scarring of any kind or cause; keloids; chloracne; acne; wrinkled skin; aging skin; oxidative stress of skin; sunburn; photodamage; skin barrier protection; skin barrier photoprotection; skin cancer; psoriasis; vitiligo; allergic dermatitis; atopic dermatitis; inflammatory skin conditions of any kind or cause; wound healing; radiation-induced dermal inflammation; fibrosis; hyperkeratosis; itching; and/or aphthous ulcers in an individual, comprising the step of administering to the individual an effective amount of one or more proton pump inhibitors.

2. The method of claim 1, wherein the cancer therapy comprises chemotherapy, radiation, surgery, one or more hormones, one or more tyrosine kinase inhibitors, one or more monoclonal antibodies, one or more immune checkpoint inhibitors or a combination thereof.

3. The method of claim 1, wherein the administering occurs systemically or locally.

4. The method of claim 1, wherein the proton pump inhibitors are formulated alone or in combination with one or more other agents.

5. The method of claim 4, wherein the one or more other agents comprises one or more cancer drugs, one or more corticosteroids, one or more antibiotics, zinc, Amifostine, Silver leaf nylon dressing, one or more pain relievers, or a combination thereof.

6. The method of claim 1, wherein the proton pump inhibitors are formulated for local administration.

7. The method of claim 1, wherein the proton pump inhibitors are formulated for topical administration.

8. The method of claim 1, wherein the proton pump inhibitors are formulated for administration outside of the alimentary canal.

9. The method of claim 1, wherein the proton pump inhibitors are formulated for administration other than for the stomach.

10. The method of claim 6, wherein the local administering is to the lungs, mucous membrane, skin, rectum, intestine, esophagus and/or blood vessels.

11. The method of claim 1, wherein the one or more proton pump inhibitors are formulated as a liquid, troche, suppository, cream, solid, tablet, pill, aerosol, gel, film, foam, ointment, paste, cream, gel, powder, drops, suspension, or combination thereof.

12. The method of claim 1, wherein the one or more proton pump inhibitors are administered prior to, during, and/or subsequent to administration of chemotherapy, radiation therapy, or both.

13. The method of claim 1, wherein a proton pump inhibitor is Omeprazole, Lansoprazole, Dexlansoprazole, Esomeprazole, Pantoprazole, Rabeprazole, Ilaprazole, or a combination thereof.

14. The method of claim 1, wherein the chemotherapy is bleomycin, carboplatin, cisplatin, doxorubicin, etoposide, mitomycin, cetuximab, gemcitabine, capecitabine, 5-fluorouracil, paclitaxel, or a combination thereof.

15. The method of claim 1, further comprising the step of administering chemotherapy, radiation, or both.

16. The method of claim 1, wherein the concentration of proton pump inhibitor is in a range of 1%-100% w/w.

17. The method of claim 1, wherein the concentration of proton pump inhibitor is not greater than 20% w/w.

18. The method of claim 1, wherein the individual has or is at risk for chemotherapy- and/or radiation therapy-induced tissue inflammation, dermatitis, scarring, and/or dermal fibrosis and wherein the proton pump inhibitor is Esomeprazole that is topically formulated.
